(19) United States
(12) Patent Application Publication
(10) Pub. No.: US 2005/0123596 A1
(43) Pub. Date: Jun. 9, 2005

(54) pH-triggered microparticles

(76) Inventors: Daniel S. Kohane, Newton, MA (US); Daniel G. Anderson, Framingham, MA (US); Robert S. Langer, Newton, MA (US); William Nicholas Haining, Newton, MA (US); Lee M. Nadler, Newton, MA (US)

Correspondence Address: CHOATE, HALL & STEWART LLP, EXCHANGE PLACE, 53 STATE STREET, BOSTON, MA, 02109, US

(21) Appl. No.: 10/948,981

(22) Filed: Sep. 23, 2004

Related U.S. Application Data
(60) Provisional application No. 60/505,355, filed on Sep. 23, 2003.
(60) Provisional application No. 60/526,481, filed on Dec. 2, 2003.

(51) Int. Cl. A61K 9/127; C12N 15/88; A61K 48/00; A61K 9/14

(52) U.S. Cl. 424/450; 424/489; 514/44; 435/458

(57) ABSTRACT

Microparticles that are designed to release their payload when exposed to acidic conditions are provided as a vehicle for drug delivery. Any therapeutic, diagnostic, or prophylatic agent may be encapsulated in a lipid-protein-sugar or polymeric matrix including a pH triggering agent to form pH triggerable microparticles. Preferably the diameter of the pH triggered microparticles ranges from 50 nm to 10 micrometers. The matrix of the particles may be prepared using any known lipid (e.g., DPPC), protein (e.g., albumin), or sugar (e.g., lactose). The matrix of the particles may also be prepared using any synthetic polymers such as polyesters. Methods of preparing and administering the particles are provided. Methods of immunization, transfection, and gene therapy are also provided by administering pH triggerable microparticles.

### RELATED APPLICATIONS

The present application claims priority under 35 U.S.C. § 119(e) to provisional application, U.S. Ser. No. 60/505,355, filed Sep. 23, 2003, entitled “pH-Triggered Microparticles,” which is incorporated herein by reference. The subject matter of the present application is also related to the subject matter disclosed in provisional application, U.S. Ser. No. 60/526,481, filed Dec. 2, 2003, entitled “pH Triggerable Polymeric Particles,” which is incorporated herein by reference.

### GOVERNMENT SUPPORT

The work described herein was supported, in part, by grants from the National Institutes of Health (GM00684-01; GM26698). The United States government may have certain rights in the invention.

### BACKGROUND OF THE INVENTION

The delivery of a drug to a patient with controlled-release of the active ingredient has been an active area of research for decades and has been fueled by the many recent developments in polymer science and the need to deliver more labile pharmaceutical agents such as nucleic acids, proteins, and peptides. Biodegradable particles have been developed as sustained release vehicles used in the administration of small molecule drugs as well as protein and peptide drugs and nucleic acids (Langer Science 249:1527-1533, 1990; Mulligan Science 260:926-932, 1993; Eldridge Mol. Immunol. 28:287-294, 1991; each of which is incorporated herein by reference). The agent to be delivered is typically encapsulated in a polymer matrix which is both biodegradable and biocompatible. As the polymer is degraded and/or as the drug diffuses out of the polymer, the agent is released into the body. Typical polymers used in preparing these particles are polyesters such as poly(glycolide-co-lactide) (PLGA), polyglycolic acid, poly-β-hydroxybutyrate, and polyacrylic acid ester. These particles have the additional advantage of protecting the agent from degradation by the body. These particles depending on their size, composition, and the agent being delivered can be administered to an individual using any route available.

In addition to the many advances in drug delivery, advances in the field of cellular immunology have allowed the identification of antigenic epitopes in many human pathogens and tumors (Rosenberg S A: A new era for cancer immunotherapy based on the genes that encode cancer antigens. Immunity 1999; 10: 281-7; Berzofsky J A, Ahlers J D, Belyakov I M: Strategies for designing and optimizing new generation vaccines. Nat Rev Immunol 2001; 1: 209-19; each of which is incorporated herein by reference). However, vaccines comprising recombinant proteins or peptides corresponding to these newly discovered epitopes often fail to induce clinically effective cell-mediated immunity. Cell-mediated immunity has been shown to be important in combating diseases such as HIV and cancer. Traditional vaccines typically prevent disease through the induction of humoral immunity. Efforts to improve the efficacy of these vaccines at inducing cell-mediated immunity have focused on enhancing the adjuvant effect of materials co-administered with the recombinant protein or peptide antigens.

Controlled release drug delivery technology has been employed by many investigators to improve the delivery of vaccine antigens to antigen-presenting cells. In particular, microparticles have been used extensively with varying degrees of success (Hanes J, Cleland J L, Langer R: New advances in microsphere-based single-dose vaccines. Adv Drug Deliv Rev 1997; 28: 97-119; incorporated herein by reference). However, one problem with the polymeric biomaterials that these microparticles are made of is their slow degradation. Even when these particles are small and are made of a polymer type and composition that is expected to degrade relatively rapidly, they can still be found in situ in profusion weeks after injection. This slow degradation may lead to sub-optimal intracellular delivery of the antigenic payload.

There remains a need for a drug delivery vehicle that allows for the rapid release of the active agent inside a cell to better target the delivery of the active agent to the site of action.

### SUMMARY OF THE INVENTION

The present invention provides a system for delivering an agent encapsulated in a microparticle that includes a pH triggering agent. The microparticles containing a pH triggering agent release their encapsulated agent when exposed to an acidic environment such as in the phagosome or endosome of a cell that has taken up the particles, thereby allowing for efficient delivery of the agent intracellularly. Typically, the pH triggering agent is a chemical compound including polymers with a pKa less than 7. As the pH triggering agent becomes protonated at the lower pH, the microparticle disintegrates thereby releasing its payload. The encapsulated agent to be delivered by the pH-triggered particles may be a diagnostic, prophylactic, or therapeutic agent. In a preferred embodiment, the agent is encapsulated in a polymeric matrix (e.g., PLGA) which includes a pH triggering agent. In other embodiments, the agent is encapsulated in a matrix of protein, sugar, and lipid that also includes a pH triggering agent. Preferably, the polymeric component or lipid-sugar-protein component of the microparticles is biocompatible and/or biodegradable. Typically the size of these particles ranges from 5 micrometers to 50 nanometers. Preferably, the microparticles are of a size that can be taken up (e.g., via phagocytosis or endocytosis) by the cells which are the target of the agent being delivered. For example, the microparticles designed to deliver antigenic peptides or proteins may have diameters in the micrometer range to allow antigen-presenting cells to take up the particles. Once taken up, the microparticles disintegrate in the acidic environment of the endosome or phagosome thereby releasing the antigenic peptide or protein inside the cell.

In certain embodiments, the pH-triggered lipid-protein-sugar particles (LPSP) typically comprise a surfactant or phospholipid or similar hydrophic or amphiphilic molecule; a protein; a simple and/or complex sugar; the agent to be delivered; and a pH triggering agent. In a particularly preferred embodiment, the lipid is dipalmitoylphosphatidylcholine (DPPC), the protein is albumin, and the sugar is lactose. In another particularly preferred embodiment, a synthetic polymer is substituted for at least one of the components of the LPSPs-lipid, protein, and/or sugar. In other embodiments, the encapsulating matrix is composed of just two components of lipid, protein, sugar, and synthetic polymer in addition to the pH triggering agent. One advantage of LPSPs over other polymeric vehicles is that the compounds used to create LPSPs are naturally occurring and therefore have improved biocompatibility compared to other polymers such as PLGA. The pH-triggered LPSPs may be prepared using any techniques known in the art including spray drying.

In another aspect, the invention provides pharmaceutical compositions comprising pH-triggered microparticles. The inventive pharmaceutical compositions may include excipients. The excipients may bulk up the microparticles, stabilizes the microparticles, make the microparticles suitable for a certain mode of administration, etc. In pharmaceutical compositions used for vaccination, the microparticles may be combined with an adjuvant to enhance the immune response. In certain embodiments, the pharmaceutical compositions include an effective amount of the microparticles to generate the desired biological response (e.g., immunize the recipient).

In another aspect, the present invention provides a method of administering the inventive pH-triggered microparticles and pharmaceutical compositions comprising pH-triggered microparticles to an individual human or animal. The pH-triggered microparticles once prepared can be administered to the individual by any means known in the art including, for example, intravenous injection, intradermal injection, rectally, orally, intravaginally, inhalationally, mucosal delivery, etc. Preferably, administration of the encapsulated agent provides release of the agent intracellularly.

In yet another aspect, the present invention provides a method of administering an antigenic epitope of a pathogen or tumor. The agent to be delivered may be a protein or peptide with at least one antigenic epitope, or it may be a nucleic acid that encodes a protein with at least one antigenic epitope. Preferably, the pH triggered microparticles are administered so that antigen-presenting cells will take up the particles. In certain embodiments, the microparticles for vaccination are delivered as a pharmaceutical composition that includes an adjuvant. The microparticles of the present invention are also useful in transfecting cells and gene therapy.

### Definitions

“Adjuvant”: The term adjuvant refers to any compound which is a nonspecific modulator of the immune response. In certain preferred embodiments, the adjuvant stimulates the immune response. Any adjuvant may be used in accordance with the present invention. A large number of adjuvant compounds are known; a useful compendium of many such compounds is prepared by the National Institutes of Health and can be found on the world wide web (see Allison Dev. Biol. Stand. 92:3-11, 1998; Unkeless et al. Annu. Rev. Immunol. 6:251-281, 1998; and Phillips et al. Vaccine 10:151-158,1992, each of which is incorporated herein by reference). Adjuvants may include lipids, oils, proteins, polynucleotides, DNAs, DNA-protein hybrids, DNA-RNA hybrids, lipoproteins, aptamers, and antibodies.

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). In certain embodiments, the animal is a human. In other embodiments, the animal is a domesticated animal (e.g., dog, cat). An animal may be a transgenic animal.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc. For example, a targeting agent may be associated with the pH triggered microparticles by non-specific interactions between the targeting agent and the surface of the microparticles.

“Biocompatible”: The term “biocompatible”, as used herein is intended to describe compounds that are not toxic to cells. Compounds are “biocompatible” if their addition to cells in vitro results in less than or equal to 20% cell death and do not induce inflammation or other such adverse effects in vivo.

“Biodegradable”: As used herein, “biodegradable” compounds are those that, when introduced into cells, are broken down by the cellular machinery into components that the cells can either reuse or dispose of without significant toxic effect on the cells (i.e., fewer than about 20% of the cells are killed, more preferably less than 10% of the cells are killed).

“Effective amount”: In general, the “effective amount” of an active agent or microparticles refers to the amount necessary to elicit the desired biological response. As will be appreciated by those of ordinary skill in this art, the effective amount of microparticles may vary depending on such factors as the desired biological endpoint, the agent to be delivered, the composition of the encapsulating matrix, the target tissue, toxicity of the agent to be delivered, the subject, etc. For example, the effective amount of microparticles containing an antigen to be delivered to immunize an individual is the amount that results in an immune response sufficient to prevent infection with an organism having the administered antigen. In another example, the effective amount of microparticles containing a tumor antigen to be delivered to immunize an individual is the amount that results in an immune response sufficient to decrease the growth of the tumor or shrink the tumor.

“Lipid”: According to the present invention, a “lipid” is any chemical compound with a hydrophobic portion. Lipids may include any surfactants, fatty acids, monoglycerdies, diglycerides, triglycerides, or hydrophobic molecules. Examples of lipids include omega-3 fatty acids, laurate, myristate, palmitate, palmitoleate, stearate, arachidate, behenate, lignocerate, palmitoleate, oleate, linoleate, linolenate, arachidonate, cholesterol, dipalmitoylphosphatidylcholine (DPPC), sphingomyelin, cerebroside, phosphoglycerides, glycolipid, etc.

“Peptide” or “protein”: According to the present invention, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide. A protein may be part of the matrix of the pH triggered microparticles encapsulating the agent to be delivered, and/or a protein may be the agent being delivered.

“Polynucleotide” or “oligonucleotide”: Polynucleotide or oligonucleotide refers to a polymer of nucleotides. Typically, a polynucleotide comprises at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Small molecule”: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Known naturally-occurring small molecules include, but are not limited to, penicillin, erythromycin, taxol, cyclosporin, and rapamycin. Known synthetic small molecules include, but are not limited to, ampicillin, methicillin, sulfamethoxazole, and sulfonamides.

“Sugar”: The term “sugar” refers to any carbohydrate. Sugars useful in the present invention may be simple or complex sugars. Sugars may be monosaccharides (e.g., dextrose, fructose, inositol), disaccharides (e.g., sucrose, saccharose, maltose, lactose), or polysaccharides (e.g., cellulose, glycogen, starch). Sugars may be obtained from natural sources or may be prepared synthetically in the laboratory. Sugars may also be obtained from natural sources and chemically modified before use. In a preferred embodiment, sugars are aldehyde- or ketone-containing organic compounds with multiple hydroyxl groups.

“Surfactant”: Surfactant refers to any agent which preferentially absorbs to an interface between two immiscible phases, such as the interface between water and an organic solvent, a water/air interface, or an organic solvent/air interface. Surfactants usually possess a hydrophilic moiety and a hydrophobic moiety, such that, upon absorbing to microparticles, they tend to present moieties to the external environment that do not attract similarly-coated particles, thus reducing particle agglomeration. Surfactants may also promote absorption of a therapeutic or diagnostic agent and increase bioavailability of the agent. The term surfactant may be used interchangeably with the terms lipid and emulsifier in the present application. Surfactants may also be used in the preparation of a pharmaceutical composition of the present invention.

### BRIEF DESCRIPTION OF THE DRAWING

FIG. 1 is a scanning electron micrograph of a 20% (w/w) Eudragit E100 particle containing 0.2% (w/w) FITC-albumin. The bar represents 5 microns.

FIG. 2 shows representative time courses of pH-triggered release of FITC-albumin from particles containing various percentages (w/w) of Eudragit E100 in phosphate-buffered saline. Arrow indicates change from pH 7.4 to pH 5. The 0% E100 particles are composed of DPPC, albumin, and lactose, as described in Example 2.

FIG. 3 includes representative times courses showing prolonged release and triggerability of FITC-albumin from 20% (w/w) Eudragit E100 particles. Arrows indicate a change from pH 7.4 to pH 5. Particles were exposed to pH 5 either 100 hours (closed box) or 390 (open circles) after initial placement in suspension.

FIG. 4 shows representative time courses showing release of Rho-lactalbumin (Rh) from particles containing various percentages (w/w) of Eudragit E100. Arrows indicate a change from pH 7.4 to pH 5. Particles were exposed to pH 5 either 4 hours (solid symbols) or 99 hours (open symbols) after initial placement in suspension.

FIG. 5 shows representative time courses showing prolonged release and triggerability of 20% (w/w) Eudragit E100 particles containing increased loading (w/w) with FITC-albumin. Arrows indicated a change from pH 7.4 to pH 5.

FIG. 6 shows tissue reaction to 20% (w/w) Eudragit E100 particles containing 0.2% (w/w) albumin four days after injection. MP, microparticles; M, muscle; I, inflammation. A. Acute inflammatory response surrounding a pocket of microparticles. ×100. B. Macrophages laden with particles (arrows). C. Edematous muscle with separated fibers adjacent to a pocket of microparticles.

FIG. 7 is a scanning electron micrograph of 20% (w/w) microparticles containing 0.2% (w/w) M58 peptide. The bar represents 5 μm.

FIG. 8 shows representative time courses of pH-triggered release of AMC-labeled M58 peptide from 20% (w/w) E100 (A) or poly-HEME (B) microparticles. Arrows indicate the time point at which the suspending medium was changed from pH 7.4 to pH 5, either 1.5 h (filled symbols) or 4 days (open symbols) after initial placement in suspension.

FIG. 9 demonstrates the selective uptake of microparticles by human APCs. Human PMBC were cultured in the presence (open histogram) or absence (gray histogram) of FITC-albumin-containing microparticles, and the percentage of cells labeled with FITC was determined using flow cytometry by gating on CD3⁺ (left panel), CD19⁺ (middle panel), or CD 14⁺ cells (right panel).

FIG. 10 is fluorescence microscopy of DCs cultured with microparticles. Human DCs were incubated for 1 hour at 37° C. (A-C) or 4° C. (D-F) with microparticles containing rhodamine-lactalbumin (red), washed extensively, and then stained to demarcate the actin cytoskeleton (green). Panels show DCs (A and D), particles (B and E), or overlaid images (C and F). G. Deconvolution fluorescence microscopy of a single DC containing rhodamine-lactalbumin microparticles after incubation at 37° C. Actin cytoskeleton is stained green and the nucleus blue.

FIG. 11 shows the time-course of phagocytosis of a microparticle (filled arrow) by an immature DC (leading edge, open arrows) visualized with time-lapse video microscopy. Representative images from the indicated times are shown.

FIG. 12 shows the effect of microparticles on DC viability, phenotype, and function. A. Apoptosis in DCs that had been cultured overnight with microparticles was assessed by annexin-V staining. Background apoptosis of DCs cultured in medium alone was subtracted. Data are representative of two separate experiments with DC from different donors. B. Cell surface expression of markers of activation/maturation on DCs after 48 hours in culture with microparticles (red histogram), poly(I:C) (green histogram), or medium control (blue histogram). Results are representative of four experiments with different donors. C. Ability of DCs to stimulate allogeneic T cell following culture with FITC-albumin-containing microparticles (closed circles) or with FITC-albumin alone (open circles) was assessed by [³H]-thymidine incorporation. Results show mean and standard deviation of proliferation measured in triplicate for three different T cell donors (50,000 cells/well) cultured for 5 days with the indicated number of DCs per well.

FIG. 13 shows the uptake of soluble or microparticle-encapsulated FITC-albumin. DCs were cultured with FITC-albumin containing microparticles (filled symbols/bars) or soluble FITC-albumin (open symbols/bars), and the frequency (A) and intensity of fluorescence (B) measured by flow cytometry. Free particles were excluded by gating based on size and CD45 staining. Data are representative of three separate experiments with DCs from different donors.

FIG. 14 shows the effect of microparticle encapsulation on antigen presentation. HLA-A*0201⁺ DCs were cultured with unencapsulated MP58 peptide (open bars) at the concentrations indicated, or with 5 μg/ml microparticles containing 0.2% or 0.02% (w/w) MP58 particles (black bars). The amount of particles added was calculated to yield concentrations of MP58 peptide equivalent to 10⁻² μg/mL or 10⁻³ g/mL, respectively. DCs were plated at 50,000 cells/well with 5,000 cells of an M58-specific clone in an IFN-γ ELISPOT assay. Results show the mean and standard deviations of triplicate measurements, and are representative of four different experiments with DCs from different donors.

FIG. 15 shows the effect of pH triggering on peptide presentation. HLA-A*0201⁺ DCs were cultured with 5 μg/mL pH-triggerable E100 particle (black bars) or nontriggerable poly-HEME (open bars) containing 0.2% (w/w) MP58, and then harvested and plated at a range of cells/well with 5000 cells of an MP58-specific clone in an IFN-γ ELISPOT assay.

FIG. 16 shows the priming of MP58-specific CTL in vivo by vaccination. HHD mice (n=5 per group) were vaccinated with equivalent amounts of MP58 encapsulated in microparticles (filled symbols) or dissolved in PBS (open symbols), and on day 7 their spleen cells were harvested and restimulated in vitro with 10 μg/mL MP58 peptide. CTL activity was tested six days later against ⁵¹Cr-labelled RMAS/HHD targets pulsed with MP58 at each of three effector:target (E:T) ratios. CTL activity against targets pulsed with irrelevant peptide was negligible. Results show the mean and standard deviation of results from each group and are representative of three separate experiments.

### DETAILED DESCRIPTION OF CERTAIN PREFERRED EMBODIMENTS OF THE INVENTION

The present invention provides a drug delivery system including microparticles that comprise a pH-triggering agent to allow for release of the active agent or payload in response to a change in pH. The present invention also provides a pharmaceutical composition with the inventive microparticles as well as methods of preparing and administering the pH-triggerable microparticles and pharmaceutical compositions. Agents administered using the pH-triggerable particles may be administered to any animal to be treated, diagnosed, or prophylaxed. The matrix of the inventive microparticles are preferably substantially biocompatible and preferably cause minimal undesired inflammatory reaction, and the degradation products are preferably easily eliminated by the body (i.e., the components of the matrix are biodegradable).

Agent

The agents to be delivered by the system of the present invention may be therapeutic, diagnostic, or prophylactic agents. Any chemical compound to be administered to an individual may be delivered using pH-triggerable microparticles. The agent may be a small molecule, organometallic compound, nucleic acid, protein, peptide, metal, an isotopically labeled chemical compound, drug, vaccine, immunological agent, etc.

In a preferred embodiment, the agents are organic compounds with pharmaceutical activity. In another embodiment of the invention, the agent is a clinically used drug that has been approved by the FDA. In a particularly preferred embodiment, the drug is an antibiotic, anti-viral agent, anesthetic, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, non-steroidal anti-inflammatory agent, nutritional agent, etc.

The agents delivered may also be a mixture of pharmaceutically active agents. For example, two or more antibiotics may be combined in the same microparticle, or two or more anti-neoplastic agents may be combined in the same microparticle. To give but another example, an antibiotic may be combined with an inhibitor of the enzyme commonly produced by bacteria to inactivate the antibiotic (e.g., penicillin and clavulanic acid).

Diagnostic agents include gases; commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium. Examples of materials useful for CAT and x-ray imaging include iodine-based materials.

Prophylactic agents include vaccines. Vaccines may comprise isolated proteins or peptides, inactivated organisms and viruses, dead organisms and viruses, genetically altered organisms or viruses, and cell extracts. Vaccines may also include polynucleotides which encode antigenic protein or peptides. Prophylactic agents may be combined with interleukins, interferon, cytokines, and adjuvants such as cholera toxin, alum, Freund's adjuvant, etc. Prophylactic agents include antigens of such bacterial organisms as Streptococccus pnuemoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorferi, Camphylobacter jejuni, and the like; antigens of such viruses as smallpox, influenza A and B, respiratory syncytial virus, parainfluenza, measles, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan, and parasitic organisms such as Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like. These antigens may be in the form of whole killed organisms, peptides, proteins, glycoproteins, carbohydrates, or combinations thereof. More than one antigen may be combined in a particular microparticle, or a pharmaceutical composition may include microparticles each containing different antigens or combinations of antigens. Adjuvants may also be combined with an antigen in the micorparticles. Adjuvants may also be included in pharmaceutical compositions of the pH triggered microparticles of the present invention.

As would be appreciated by one of skill in this art, the variety and combinations of agents that can be delivered using the pH triggered microparticles are almost limitless. The pH triggered microparticles find particular usefulness in delivering agents to an acidic environment or into cells. In certain embodiments, the microparticles are designed to deliver agents to a tumor. In other embodiments, the microparticles are designed to deliver agents to cells of the immune system such as antigen-presenting cells (APCs), dendritic cells, monocytes, and macrophages.

pH Triggering Agent

The pH triggering agents useful in the present invention are any chemical compounds that lead to the destruction, degradation, or dissolution of a microparticle containing the pH triggering agent in response to a change in pH, for example, a decrease in pH. In certain embodiments, the pH triggering agent may degrade in response to an acidic pH (e.g., acid hydrolysis of ortho-esters). In other embodiments, the pH triggering agent may dissolve or become more soluble at an acidic pH. The pH triggering agents useful in the present invention may include any chemical compound with a pKₐ between 3 and 7. Preferably the pKₐ of the triggering agent is between 5 and 6.5. In certain embodiments, the pH triggering agent is insoluble or substantially insoluble at physiologic pH (i.e., 7.4), but water soluble at acidic pH (i.e., pH<7, preferably, pH<6.5). Without being bound by any particular theory, the pH sensitivity of the microparticles containing a pH triggering agent stems from the fact that the pH triggering agent within the matrix of the microparticles become protonated when exposed to a low pH environment. This change in state of protonation causes the pH triggering agent to become more soluble in the surrounding environment, and/or the change in protonation state disrupts the integrity of the matrix of the microparticle causing it to fall apart. When the triggering agent dissolves or the microparticle is disrupted, the agent contained within the microparticle is released. The pH triggered microparticles are particularly useful in delivering agents to acidic environments such as the phagosomes or endosomes of cells.

The pH triggering agent may be a small molecule or a polymer. In certain preferred embodiments, the pH triggering agent is a polymer with a pKₐ between 5 and 6.5. In certain embodiments, the pH triggering agent has nitrogen-containing functional groups such as amino, alkylamino, dialkylamino, arylamino, diarylamino, imidazolyl, thiazolyl, oxazolyl, pyridinyl, piperidinyl, etc. Certain preferred polymers include polyacrylates, polymethacrylates, poly(beta-amino esters), and proteins. In certain embodiments, the pH triggering agent is Eudragit E100 (poly(butyl methacrylate-co-(2-dimethylaminoethyl) methacrylate-co-methyl methacrylate (1:2:1)). In other embodiments, the pH triggering agent is a polymer that is soluble in an acidic aqueous solution. In other embodiments, the pH triggering agent is a cationic protein at physiological pH (pH 7.4). pH triggering agents may also be lipids or phospholipids.

The pH triggering agents may comprise 1-80% of the total weight of the microparticle. In certain embodiments, the weight:weight percent of the pH triggering agent is less than or equal to 40%, more preferable less than or equal to 20%, and most preferably, ranging from 1-5%.

The pH triggering agent is preferably part of the matrix of the microparticle. The pH triggering agent may be associated with the components of the matrix through covalent or non-covalent interactions. In certain embodiments, the pH triggering agent will be dispersed throughout the matrix of the particle. In other embodiments, the pH triggering agent may only be found in a shell of the microparticle and will not be dispersed throughout the particle. The shell may be an outer shell, an inner shell, or a shell within the matrix. For example, the pH triggering agent may only be found on the inside of the particle.

Microparticle Matrix

The agent is encapsulated in a matrix to form microparticles. Any material known in the art to be useful in preparing microparticles may be used in preparing pH-triggerable microparticles. The pH-triggering agent is typically incorporated into the matrix of the microparticle. The matrix may include a natural or synthetic polymer, or a blend or mixture of polymers. In other embodiments, the matrix is a lipid-protein-sugar matrix as described in U.S. Ser. No. 09/981,020, filed Oct. 16, 2001, and U.S. Ser. No. 09/981,460, filed Oct. 16, 2001; each of which is incorporated herein by reference. Other preferred embodiments include a lipid-protein matrix, a lipid-sugar matrix, or a protein-sugar matrix. In certain embodiments, the lipid, protein, or sugar component of the matrix may be replaced with a synthetic polymer (e.g., poly(lactic-co-glycolic acid) (PLGA), polyglycolic acid (PGA), polyesters, polyanhydrides, polyamides, etc.).

The size of the microparticles will depend on the use of the particles. For example, an application requiring the microparticles to be phagocytosed by cells may use particles ranging from 1-10 microns in diameter, more preferably 2-6 microns in diameter. In certain preferred embodiments, the diameter of the microparticles ranges from 50 nanometers to 50 microns. In other preferred embodiments, the microparticles are less than 10 micrometers, and more preferably less than 5 micrometers. In certain embodiments, the microparticles range in size from 2-5 microns in diameter. The size of the microparticles and distribution of sizes may be selected by one of ordinary skill in the art based on the agent being delivered, the target tissue, route of administration, method of uptake by the cells, etc. The specific ratios of the excipients may range widely depending on factors including size of particle, porosity of particle, agent to be delivered, desired agent release profile, target tissue, etc. One of ordinary skill in the art may test a variety of ratios and specific components to determine the composition correct for the desired purpose.

Lipids (Surfactants or Emulsifiers)

The lipid portion of the matrix of inventive pH triggerable LPSPs is thought to bind the particle together. The hydrophobicity of the lipid may also contribute to the slow release of the encapsulated drug. In other embodiments, the lipid may contribute to the increased release of the agent (e.g., a nucleic acid). The percent of lipid in the matrix (excluding the agent) may range from 0% to 99%, more preferably from 3% to 99%. In certain preferred embodiments, the weight percent of lipid in the microparticle ranges from 20% to 80%, preferably from 50%-70%, more preferably around 60%. In other embodiments, the weight percent of lipid in the microparticle ranges from 5-20%, more preferably from 10-15%, more preferably around 10%.

Any lipid, surfactant, or emulsifier known in the art is suitable for use in making the inventive microparticles. Such surfactants include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid amides; sorbitan trioleate (Span 85) glycocholate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecylamine; acetyl palmitate; glycerol ricinoleate; hexadecyl sterate; isopropyl myristate; tyloxapol; poly(ethylene glycol)5000-phosphatidylethanolamine; and phospholipids. The lipid component may also be a mixture of different lipid molecules. These lipid may be extracted and purified from a natural source or may be prepared synthetically in a laboratory. In a preferred embodiment, the lipids are commercially available.

Protein

The protein component of the encapsulating matrix may be any protein or peptide. The protein of inventive pH triggerable LPSPs presumably plays a structural role in the microparticles. Proteins useful in the inventive system include albumin, gelatin, whole cell extracts, antibodies, and enzymes (e.g., glucose oxidase, etc.). The protein may be chosen based on known interactions between the protein and the agent being delivered. For example, bupivacaine is known to bind to albumin in the blood; therefore, albumin would be a logical choice in choosing a protein from which to prepare microparticles containing bupivacaine. In certain embodiments, the protein of the matrix may be the actual agent being delivered, for example, an antigenic protein may function as the protein in the LPSP and be the agent to be delivered. The percentage of protein in the matrix (excluding the agent to be delivered) may range from 0% to 99%, more preferably 1% to 80%, and most preferably from 10% to 60%. In certain embodiments, the percent of protein in the microparticle is approximately 10%, 20%, 30%, 40%, 50%, 60%, 70%, 80%, or 90%, preferably approximately 20%.

In certain preferred embodiments, the agent to be delivered is a protein. In these embodiments, the protein to be delivered may make up all or a portion of the protein component of the encapsulating matrix. Preferably, the protein maintains a significant portion of its original activity after having been processed to form microparticles.

In another particularly preferred embodiment, at least a portion of the protein is immunoglobulins. These immunoglobulins may serve as a targeting agent. For example, the binding site of the immuoglobulin may be directed to an epitope normally found in a tissue or on the cell surface of cells being targeted (e.g., tumor cells, bacteria, fungi). The targeting of a specific receptor may lead to endocytosis or phagocytosis of the microparticle. For example, the antibody may be directed to the LDL receptor.

The protein component may be provided using any means known in the art. In certain preferred embodiments, the protein is commercially available. The protein may also be purified from natural or recombinant sources, or may be chemically synthesized. In certain preferred embodiments, the protein has been purified and is greater than 75% pure, more preferably greater than 90% pure, even more preferably greater than 95% pure, most preferably greater than 99% or even 100% pure.

Sugar

The sugar component of inventive pH triggerable LPSPs may be any simple or complex sugar. The sugar component of the matrix is thought to play a structural role in the particles and may also lead to increased biocompatibility. The percent of sugar in the matrix excluding the agent can range from 0% to 99%, more preferably from approximately 0.5% to approximately 50%, and most preferably from approximately 10% to approximately 40%. In certain embodiments, the percentage of sugar is approximately 5%, 10%, 20%, 30%, 40%, 50%, 60%, 70%, 80%, or 90%, preferably 20%.

Natural as well as unnatural sugars may be used in the inventive microparticles. Sugars that may be used in the present invention include, but are not limited to, galactose, lactose, glucose, maltose, starches, cellulose and its derivatives (e.g., methyl cellulose, carboxymethyl cellulose, etc.), fructose, dextran and its derivatives, raffinose, mannitol, xylose, dextrins, glycosaminoglycans, sialic acid, chitosan, hyaluronic acid, and chondroitin sulfate. Preferably, the sugar component like the protein and lipid components is biocompatible and/or biodegradable. In certain preferred embodiment, the sugar component is a mixture of sugars.

The sugar may be from natural sources or may be synthetically prepared. Preferably, the sugar is available commerically.

In a particularly preferred embodiment, the sugar of the matrix may also function as a targeting agent. For example, the ligand of a receptor found on the cell surface of cells being targeted or a portion of the ligand may be the same sugar in the microparticle or may be similar to the sugar in the microparticle, or the sugar may also be designed to mimic the natural ligand of the receptor.

Polymers

Any polymer may be used in preparing the pH triggered particles of the present invention. As described above a polymer may substitute for any one or two of the other components in LPSPs. In other embodiments, the polymer and pH triggering agent alone form the matrix of the inventive microparticle. For example, a microparticle may include an agent encapsulated in an PLGA matrix that includes a pH triggering agent.

The polymers useful in the present invention include natural as well as unnatural polymers. Preferably, the polymers are both biocompatible and biodegradable. Polymers useful in the present invention include polyesters, polyamides, polycarbonates, polycarbamates, polyacrylates, polystyrene, polyureas, polyethers, polyamines, etc. The polymer may make up from 1-99% of the microparticle. Preferably, the polymer is 5-80% of the microparticle. Even more preferably, the polymer is from 70-90% of the microparticle. In certain embodiment, the polymer is approximately 10%, 20%, 30%, 40%, 50%, 60%, 70%, 80%, or 90% of the microparticle excluding the agent being delivered, preferably at least 50%.

Targeting Agents

The inventive microparticles may be modified to include targeting agents since it is often desirable to target drug delivery to a particular cell, collection of cells, tissue, or organ. A variety of targeting agents that direct pharmaceutical compositions to particular cells are known in the art (see, for example, Cotten et al. Methods Enzym. 217:618, 1993; incorporated herein by reference). The targeting agents may be included throughout the particle or may be only on the surface. The targeting agent may be a protein, peptide, carbohydrate, glycoprotein, lipid, small molecule, etc. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialycoproteins, gp120 envelope protein of the human immunodeficiency virus (HIV), carbohydrates, receptor ligands, sialic acid, etc. If the targeting agent is included throughout the particle, the targeting agent may be included in the mixture that is spray dried to form the particles. If the targeting agent is only on the surface, the targeting agent may be associated with (i.e., by covalent, hydrophobic, hydrogen boding, van der Waals, or other interactions) the formed particles using standard chemical techniques.

Pharmaceutical Compositions

Once the pH triggerable microparticles have been prepared, they may be combined with other pharmaceutical excipients to form a pharmaceutical composition. As would be appreciated by one of skill in this art, the excipients may be chosen based on the route of administration as described below, the agent being delivered, time course of delivery of the agent, etc.

Pharmaceutical compositions of the present invention and for use in accordance with the present invention may include a pharmaceutically acceptable excipient or carrier. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, subcutaenously, intradermally, transdermally, intravenously, intraarterially, or as an oral or nasal spray.

Liquid dosage forms for oral administration include pharmaceutically acceptable emulsions, microemulsions, solutions, suspensions, syrups, and elixirs. In addition to the active ingredients (i.e., microparticles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In a particularly preferred embodiment, the microparticles are suspended in a carrier fluid comprising 1% (w/v) sodium carboxymethyl cellulose and 0.1% (v/v) Tween 80.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the inventive micropartilces with suitable non-irritating excipients or carriers such as cocoa butter, polyethylene glycol, or a suppository wax which are solid at ambient temperature but liquid at body temperature and therefore melt in the rectum or vaginal cavity and release the microparticles.

Solid dosage forms for oral administration include capsules, tablets, pills, powders, and granules. In such solid dosage forms, the microparticles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Dosage forms for topical or transdermal administration of an inventive pharmaceutical composition include ointments, pastes, creams, lotions, gels, powders, solutions, sprays, inhalants, or patches. The microparticles are admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams, and gels may contain, in addition to the microparticles of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the microparticles of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the microparticles in a proper medium. Absorption enhancers can also be used to increase the flux of the compound across the skin. The rate can be controlled by either providing a rate controlling membrane or by dispersing the microparticles in a polymer matrix or gel.

Methods of Making Microparticles

The inventive microparticles may be prepared using any method known in this art. These include spray drying, single and double emulsion solvent evaporation, solvent extraction, solvent evaporation, phase separation, simple and complex coacervation, and other methods known to those of skill in the art (see, e.g., U.S. Pat. Nos. 6,740,310; 6,652,837; 6,254,890; 6,007,845; 5,912,017; 5,783,567; 5,626,862; 5,565,215; 5,543,158; 5,500,161; 5,356,630; and 4,272,398; each of which is incorporated herein by reference). A particularly preferred method of preparing the particles is spray drying. The conditions used in preparing the microparticles may be altered to yield particles of a desired size or property (e.g., hydrophobicity, hydrophilicity, external morphology, “stickiness”, shape, porosity, density, etc.). The method of preparing the particle and the conditions (e.g., solvent, temperature, concentration, air flow rate, etc.) used may also depend on the agent being encapsulated, the composition of the matrix. and/or the pH triggering agent.

Methods developed for making microparticles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al. Reactive Polymers 6:275-283, 1987; Mathiowitz et al. J. Appl. Polymer Sci. 35:755-774, 1988; each of which is incorporated herein by reference).

After the particles are prepared, additional steps may be performed to select particles of a particular size or other characteristic (e.g., shape, density, porosity, stickiness, stability, external morphology, crystallinity, loading, etc.) (Mathiowitz et al. Scanning Microscopy 4:329-340 (1990); Mathiowitz et al. J. Appl. Polymer Sci. 45:125-34 (1992); Benita et al. J. Pharm. Sci. 73:1721-24, 1984; each of which is incorporated herein by reference). If the particles prepared by any of the above methods have a size range outside of the desired range, the particles can be sized, for example, using a sieve.

As described above, pH triggerable microparticles are preferably prepared by spray drying. Prior methods of spray drying, such as those disclosed in PCT WO 96/09814 by Sutton and Johnson (incorporated herein by reference), provide the preparation of smooth, spherical microparticles of a water-soluble material with at least 90% of the particles possessing a mean size between 1 and 10 micrometers. The method disclosed by Edwards et al. in U.S. Pat. No. 5,985,309 (incorporated herein by reference) provides rough (non-smooth), non-spherical microparticles that include a water-soluble material combined with a water-insoluble material. Any of the methods described above may be used in preparing the inventive microparticles. Specific methods of preparing microparticles are described below in the Examples.

Administration

The pH triggerable microparticles and pharmaceutical compositions containing the inventive microparticles may be administered to an individual via any route known in the art. These include, but are not limited to, oral, sublingual, nasal, intradermal, subcutaneous, intramuscular, rectal, vaginal, intravenous, intraarterial, transdermal, intradermal, and inhalational administration. In certain embodiments, the microparticles are delivered to a mucosal surface. As would be appreciated by one of skill in this art, the route of administration and the effective dosage to achieve the desired biological effect is determined by the agent being administered, the target organ, the preparation being administered, time course of administration, disease being treated, etc.

The inventive microparticles are also useful in the transfection of cells making them useful in gene therapy. The microparticles with polynucleotides to be delivered are contacted with cells under suitable conditions to have the polynucleotide delivered intracellularly. Conditions useful in transfection may include adding calcium phosphate, adding a lipid, adding a lipohilic polymer, sonication, etc. The cells may be contacted in vitro or in vivo. Any type of cells may be transfected using the pH triggered microparticles. In certain embodiments, the microparticles are administered inhalationally to delivery a polynucleotide to the lung epithelium of a patient. This method is useful in the treatment of hereditary diseases such as cystic fibrosis.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### pH-Triggered Microparticles Enhance Peptide Antigen Delivery to Dendritic Cells: Implications for Tumor Vaccines

Despite the presence of tumor-specific T cells in many cancer patients, most tumor vaccines fail to boost tumor immunity to clinically meaningful levels. One obstacle to effective vaccination is inadequate antigen delivery to professional antigen presenting cells (APC). We therefore sought to design an antigen-delivery vehicle which would be taken up readily by APC; release vaccine antigens in acidic phago-lysosomal compartments; and protect antigens from extra-cellular degradation. Using a spray-drying method we produced 3-5 μm microparticles (MP) composed of: (1) a protein of interest; (2) the phospholipid dipalmitoylphosphatidylcholine; and (3) the polymethacrylate Eudragit, which is insoluble in water at physiological pH, but very soluble at acidic pH. A wide range of proteins and peptides were successfully encapsulated in MP. Kinetic studies showed that release of MP contents took days to weeks in phosphate-buffered saline (pH 7.4) but was immediate in acetate buffer (pH 5). To test whether MP were taken up by cells we co-cultured normal peripheral blood mononuclear cells with MP-encapsulated FITC-albumin (F-Alb) and determined the uptake of MP by different cell types using flow cytometry. Only monocytes were labeled with F-Alb, indicating that only cells capable of phagocytosis were targeted by MP. To determine the effect of MP encapsulation on F-Alb uptake by human dendritic cells (DC), we generated monocyte-derived DC and cultured them with either free or MP-encapsulated F-Alb. At equivalent protein concentrations, MP-encapsulated F-Alb labeled >60% of DC while free F-Alb that labeled <20% of DC, demonstrating that MP-encapsulation markedly enhances protein uptake. Time-lapse video microscopy showed rapid adherence of MP to DC, with resulting phagocytosis in <2 h. Uptake of MP was not toxic to DC, as it did not cause significant apoptosis, alter cell phenotype or decrease their ability to stimulate allogeneic T cells. To measure intracellular release of peptide antigen, we produced MP containing 0.2% (w/w) of an HLA-A*0201-restricted epitope (Flu) from the Influenza matrix protein, chosen to represent a typical nonamer peptide that might be used in a peptide vaccine. Delivery of antigen to DC was measured by interferon-γ release from a Flu-specific T cell clone. The clone was readily stimulated by DC co-cultured with MP-encapsulated Flu (MP-Flu), demonstrating effective intracellular delivery of the antigen. Moreover the amount of stimulation was equivalent to that caused by a concentration of free Flu peptide 1 to 2 log units greater than that present in MP-Flu, showing a significant improvement in antigen delivery by MP-encapsulation. To test antigen-delivery by MP in vivo, mice transgenic for HLA-A*0201 were given a subcutaneous injection of MP-Flu. Preliminary results showed that Flu-specific T cells could be primed by a single vaccination of MP-Flu even in the absence of adjuvant, demonstrating effective antigen delivery to APC in vivo. Such MP are attractive as delivery agents because: (1) they are biocompatible; (2) a range of compounds (e.g., adjuvants) can be co-encapsulated with antigen; and (3) their production is easy to scale up. Our data suggest that pH-triggered, controlled-release MP markedly improve the delivery of peptide antigen in vitro and in vivo and may increase the efficacy of tumor vaccines used to treat patients with cancer.

### Example 2

### pH-Triggered Release of Macromolecules from Spray-Dried Polymethacrylate Microparticles

Introduction

Microparticulate formulations for controlled release of therapeutic agents have been used to achieve both systemic and local drug delivery. However, there are a number of biomedical applications where the desired goal is enhanced delivery into an intracellular compartment. Examples include vaccination, transfection, and the treatment of infections that are located within macrophages (J. Hanes, J. L. Cleland, and R. Langer. New advances in microsphere-based single-dose vaccines. Adv Drug Deliv Rev 28: 97-119 (1997); M. L. Hedley, J. Curley, and R. Urban. Microspheres containing plasmid-encoded antigens elicit cytotoxic T-cell responses. Nat Med 4: 365-8 (1998); A. K. Agrawal, and C. M. Gupta. Tuftsin-bearing liposomes in treatment of macrophage-based infections. Adv Drug Deliv Rev 41: 135-46 (2000); incorporated herein by reference). The encapsulation of drugs in microparticles can facilitate drug delivery via two main mechanisms: 1) the payload is protected from the extracellular environment until the particle is taken up by cells, 2) uptake may be targeted to professional antigen presenting cells. Macromolecule delivery within cells can be further improved by designing microparticles so that they release their payload instantaneously in response to a low pH so that they would disintegrate following phagocytosis when exposed to the pH (5 to 6.5) in the phagosome, thereby releasing their contents inside the cell (R. Reddy, F. Zhou, L. Huang, F. Carbone, M. Bevan, and B. T. Rouse. pH sensitive liposomes provide an efficient means of sensitizing target cells to class I restricted CTL recognition of a soluble protein. J Immunol Methods 141: 157-63 (1991); O. V. Gerasimov, J. A. Boomer, M. M. Qualls, and D. H. Thompson. Cytosolic drug delivery using pH- and light-sensitive liposomes. Adv Drug Deliv Rev 38: 317-338 (1999); D. Luo, and W. M. Saltzman. Synthetic DNA delivery systems. Nat Biotechnol 18: 33-7 (2000); D. M. Lynn, M. M. Amiji, and R. Langer. pH-responsive polymer microspheres: Rapid release of encapsulated material within the range of intracellular pH. Angewandte Chemie-International Edition 40: 1707-1710 (2001); each of which is incorporated herein by reference).

We have previously described spray-dried microparticles composed of variable combinations of phospholipids, proteins, simple or complex sugars, and/or drugs with varying physicochemical properties, and have demonstrated their safety, biocompatibility, and efficacy for drug delivery to the peripheral and central nervous systems (D. S. Kohane, M. Lipp, R. Kinney, N. Lotan, and R. Langer. Sciatic nerve blockade with lipid-protein-sugar particles containing bupivacaine. Pharm. Res. 17: 1243-1249 (2000); D. S. Kohane, N. Plesnila, S. S. Thomas, D. Le, R. Langer, and M. A. Moskowitz. Lipid-sugar particles for intracranial drug delivery: safety and biocompatibility. Brain Res 946: 206-13 (2002); D. S. Kohane, G. L. Holmes, Y. Chau, D. Zurakowski, R. Langer, and B. H. Cha. Effectiveness of muscimol-containing microparticles against pilocarpine-induced focal seizures. Epilepsia 43: 1462-8 (2002); each of which is incorporated herein by reference). Particles of this type can be made to be of a size and density suitable for inhalational drug delivery (D. S. Kohane, M. Lipp, R. Kinney, N. Lotan, and R. Langer. Sciatic nerve blockade with lipid-protein-sugar particles containing bupivacaine. Pharm. Res. 17: 1243-1249 (2000); A. Ben-Jebria, D. Chen, M. L. Eskew, R. Vanbever, R. Langer, and D. A. Edwards. Large porous particles for sustained protection from carbachol-induced bronchoconstriction in guinea pigs. Pharm. Res. 16: 555-561 (1999); each of which is incorporated herein by reference). Here, we have developed an approach to rendering these microparticles pH-triggerable by incorporating a polymethacrylate (Eudragit E 100, termed E100 here) as a model pH-sensitive material, so that they can be optimized for intracellular drug delivery. E100 is insoluble in aqueous media at physiologic pH, but water soluble at acidic pH.

The non-pH triggered versions of these particles have other properties that may be desirable in this context. They are typically 2 to 5 μm in diameter, thus being of a size that should allow them to be taken up by phagocytosis by immune cells (Y. Tabata, and Y. Ikada. Phagocytosis of polymer microspheres by macrophages. Adv. Polymer Sci. 94: 107-141 (1990); incorporated herein by reference), while being too large to be taken up by cells that are not “professionally” phagocytic. Particles of this type produce a transient mild acute inflammatory response, thus potentially attracting the target cell. However, they also have excellent long-term biocompatibility (D. S. Kohane, N. Plesnila, S. S. Thomas, D. Le, R. Langer, and M. A. Moskowitz. Lipid-sugar particles for intracranial drug delivery: safety and biocompatibility. Brain Res 946: 206-13 (2002); D. S. Kohane, M. Lipp, R. Kinney, D. Anthony, N. Lotan, and R. Langer. Biocompatibility of lipid-protein-sugar particles containing bupivacaine in the epineurium. J. Biomed. Mat. Res. 59: 450-459 (2002); each of which is incorporated herein by reference), partly as a result of the fact that they can be made of excipients that occur naturally in the target milieu. The method of manufacture allows very high maximum loading of the particles with the macromolecule of interest, thus reducing the particulate mass to be injected and hence the associated tissue reaction. The fact that these particles can be easily modified to allow delivery via inhalation is also appealing in the context of the development of methods of providing mucosal immunity (L. Stevceva, A. G. Abimiku, and G. Franchini. Targeting the mucosa: genetically engineered vaccines and mucosal immune responses. Genes Immun 1: 308-15 (2000); incorporated herein by reference).

This formulation may also be desirable when other common particle production methods are not optimal, such as when co-encapsulation of certain combinations of excipients (or drugs) with differing solubilities is desired (D. S. Kohane, M. Lipp, R. Kinney, N. Lotan, and R. Langer. Sciatic nerve blockade with lipid-protein-sugar particles containing bupivacaine. Pharm. Res. 17: 1243-1249 (2000); D. S. Kohane, N. Plesnila, S. S. Thomas, D. Le, R. Langer, and M. A. Moskowitz. Lipid-sugar particles for intracranial drug delivery: safety and biocompatibility. Brain Res 946: 206-13 (2002); each of which is incorporated herein by reference), or for the production of relatively porous particles (e.g., for inhalational use). In such situations, spray drying is a useful alternative; its advantages have been reviewed (K. Keith. Spray drying handbook, John Wiley, New York, 1991; incorporated herein by reference).

In addition we describe the particles' release of fluorescein-labeled albumin (68 kd) and rhodamine-labeled lactalbumin (15 kd) in vitro. We also verify the ability of these modified particles to attract immune cells, and study their biocompatibility by injecting them at a location where there are many tissue types (muscle, nerve, connective tissue), the sciatic nerve at the hip.

Materials and Methods

Materials

Fluorescein isothiocyanate-conjugated albumin (FITC-albumin) and rhodamine-labeled lactalbumin (Rho-lactalbumin) were purchased from Sigma Chemical Co. (St. Louis, Mo.), L-alpha-dipalmitoylphosphatidylcholine (DPPC) from Avanti Polar Lipids (Alabaster, Ala.), and USP grade ethanol from Pharmco Products (Brookfield, Conn.). Eudragit E 100 (poly(butyl methacrylate-co-(2-dimethylaminoethyl) methacrylate-co-methyl methacrylate)=1:2:1 (termed E100 below) was a gift from Röhm GmbH (Darmstadt, Germany).

Production of Microparticles

Varying proportions of DPPC and E100, totaling 500 mg of solute, were dissolved in 87.5 ml of ethanol. One milligram of FITC-albumin or Rho-lactalbumin in 37.5 ml of water was added dropwise to this solution. In some experiments 5 to 100 mg of FITC-albumin were used, with a corresponding decrease in the amount of DPPC, while the amount of E100 was kept constant. For example, particles that were 20% (w/w) FITC-albumin, 20% (w/w) E100 were made by incorporating 100 mg FITC-albumin, 100 mg E100, and 300 mg DPPC. The resulting mixture was spray-dried using a Model 190 bench top spray drier (Büchi Co, Switzerland), using the following settings: air flow rate: 600 L/min, aspiration −20 mbar, solvent flow: 12 ml/min, inlet temperature: 110-120 degrees C., outlet temperature 39-48 degrees C. Particles without E100 were made with the composition 60% (w/w) DPPC, 19.8% (w/w) albumin, 20% (w/w) lactose, as previously described (D. S. Kohane, M. Lipp, R. Kinney, N. Lotan, and R. Langer. Sciatic nerve blockade with lipid-protein-sugar particles containing bupivacaine. Pharm. Res. 17: 1243-1249 (2000); D. S. Kohane, G. L. Holmes, Y. Chau, D. Zurakowski, R. Langer, and B. H. Cha. Effectiveness of muscimol-containing microparticles against pilocarpine-induced focal seizures. Epilepsia 43: 1462-8 (2002); each of which is incorporated herein by reference), with 0.2% (w/w) FITC-albumin added.

Particle Size, Shape, and Density Determination.

Particle size was determined with a Coulter Multisizer (Coulter Electronics Ltd., Luton, U.K.), using a 30-μm orifice. Surface characteristics of particles were determined by scanning electron microscopy on an AMR-1000 (Amray Inc., Bedford, Mass.). Samples were mounted on stubs and given a gold-palladium conductive coating, and scanned at 10 kV. Particle density was determined by placing a known weight of particles into a graduated tube and tapping the tube against a benchtop 50 times, after which the density was calculated as the weight divided by the volume.

Release of FITC-Albumin from Microparticles

5 mg of each particle type were suspended in 1 ml of 100 mM phosphate-buffered saline pH 7.4 (PBS), and incubated at 37 degrees C. At predetermined timepoints, the samples were centrifuged, and the supernatants removed for fluorimetry. The pellets were resuspended in PBS. After a given time point, the phosphate-buffered saline was replaced with 100 mM sodium acetate pH 5; sample treatment was otherwise unchanged.

Fluorimetry was performed on a PTI system (Photon Technology International, Lawrenceville, N.J.) at the following wavelengths: FITC-albumin excitation 485, emission: 515; Rho-lactalbumin excitation 560, emission: 584.

In Vivo Experiments

Animals were cared for in compliance with protocols approved by the Massachusetts Institute of Technology Committee on Animal Care, in conformity with the “Principles of Laboratory Animal Care” (NIH publication #85-23, revised 1985). Sprague-Dawley rats were obtained from Charles River Laboratories (Wilmington, Mass.). They were housed in groups and kept in a 6 am-6 pm light-dark cycle. Young adult male Sprague-Dawley rats weighing 310-420 g were used. Twenty-five milligrams of microparticles suspended in 0.6 ml of carrier fluid (1% (w/v) sodium carboxymethyl cellulose, 0.1% (v/v) Tween 80) were injected at the sciatic nerve under general anesthesia as described (D. S. Kohane, M. Lipp, R. Kinney, N. Lotan, and R. Langer. Sciatic nerve blockade with lipid-protein-sugar particles containing bupivacaine. Pharm. Res. 17: 1243-1249 (2000); incorporated herein by reference). Every day after injection, animals were examined for self-mutilation (D. S. Kohane, M. Lipp, R. Kinney, D. Anthony, N. Lotan, and R. Langer. Biocompatibility of lipid-protein-sugar particles containing bupivacaine in the epineurium. J. Biomed. Mat. Res. 59: 450-459 (2002); P. D. Wall, M. Devor, R. Inbal, J. W. Scadding, D. Schonfeld, Z. Seltzer, and M. M. Tomkiewicz. Autotomy following peripheral nerve lesions: experimental anaesthesia dolorosa. Pain 7: 103-111 (1979); each of which is incorporated herein by reference), a behavior believed to be pain-related, and received a neurobehavioral assessment as described (J. G. Thalhammer, M. Vladimirova, B. Bershadsky, and G. R. Strichartz. Neurologic evaluation of the rat during sciatic nerve block with lidocaine. Anesthesiology 82: 1013-1025 (1995); D. S. Kohane, J. Yieh, N. T. Lu, R. Langer, G. Strichartz, and C. B. Berde. A re-examination of tetrodotoxin for prolonged anesthesia. Anesthesiology 89: 119-131 (1998); each of which is incorporated herein by reference). In brief, thermal nociception was assessed by a modified hotplate test at 56° C. (Model 39D Hot Plate Analgesia Meter, IITC Inc., Woodland Hills, Calif.). Motor strength was assessed by holding the rat with its posterior above a digital balance and measuring the maximum weight that the rat could bear without its ankle touching the balance. One or four days after injection, the sciatic nerves and adjacent tissues were harvested (D. S. Kohane, M. Lipp, R. Kinney, D. Anthony, N. Lotan, and R. Langer. Biocompatibility of lipid-protein-sugar particles containing bupivacaine in the epineurium. J. Biomed. Mat. Res. 59: 450-459 (2002); each of which is incorporated herein by reference) under deep isoflurane anesthesia followed by pentobarbital euthanasia, embedded in paraffin and stained with hematoxylin and eosin using standard techniques. For subcutaneous injections, the same dose and volume of injectate, and animal protocol were used with the exception that the needle was inserted into the loose skin between the shoulder blades, advanced 1 cm parallel to the axis of the animal, and the particle suspension injected.

Results

Protein-Containing Particles

Particles were made as described above, containing 0%, 1%, 5%, 20%, 40%, and 80% E100 (w/w), with corresponding proportions of DPPC and an invariant amount of FITC-albumin or Rho-lactalbumin (0.2% (w/w)). Particle yields by weight were generally in the range of 20 to 40% of the total mass of solute, except for the 1% (w/w) Eudragit particles, where the yield was 10 to 20%. Particle density varied in inverse proportion to the proportion of Eudragit and protein in the formulation. Particles with 20% (w/w) or less of Eudragit were relatively dense (approximately 0.25 g/ml), while particles with 40% (w/w) Eudragit were roughly half as dense (approximately 0.13 mg/ml). Twenty percent (w/w) particles containing 20% (w/w) protein loading had densities roughly one-half those of the corresponding particles with 0.2% (w/w) protein (0.13 and 0.12 mg/ml for FITC-albumin and Rho-lactalbumin respectively).

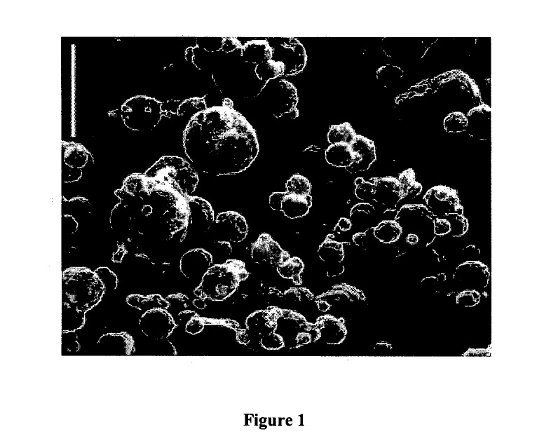

A representative scanning electron micrograph of 20% (w/w) E100 microparticles is shown in FIG. 1. In general, particles were spherical or roughly spheroidal, although some were irregular or concave. The median volume weighted particle diameters were in the range of 3 to 5 μm by Coulter counting.

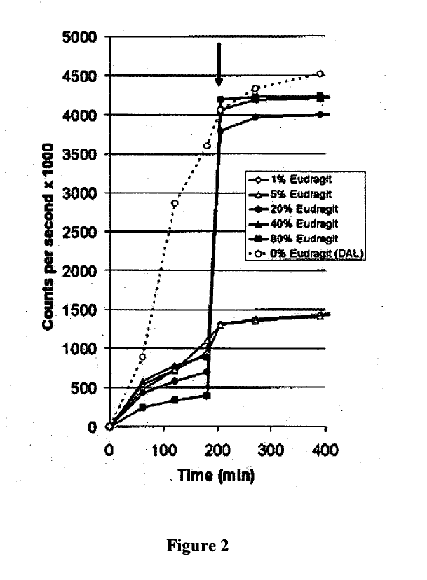

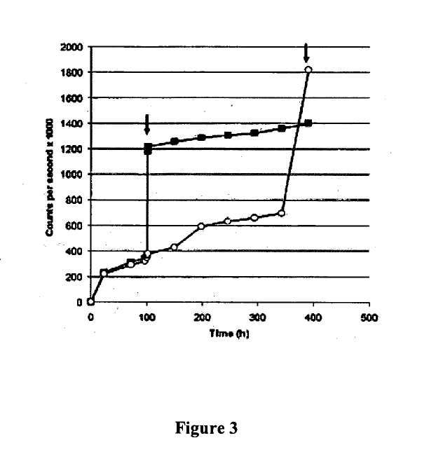

We assessed the release of FITC-albumin from the various particle types in 100 mM phosphate buffered saline, pH 7.4 at 37° C., in which particles suspended readily. Release from these particles was slow (FIGS. 2, 3), particularly compared to particles where the E100 was replaced by other excipients such as albumin and lactose (FIG. 2, 0% Eudragit). In the absence of a triggering stimulus, release proceeded for at least two weeks (341 h; FIG. 3). This was in marked contrast to the rapid release of labeled proteins from particles without E100, or particles composed of DPPC, albumin and lactose where the majority of the FITC-albumin was released within the first hour.

The effect of re-suspending the particle pellet in 100 mM sodium acetate, pH 5 depended on the proportion of E100 in the particles (FIG. 2). The suspension of particles with high proportions of E100, which was cloudy at pH 7.4, became clear at pH 5. In the case of 80% (w/w) E100 particles, there was no solid material left in the test tube after exposure to pH 5. For the other formulations, subsequent centrifugation yielded a pellet of fine white powder, whose size was in inverse proportion to the amount of E100. Particles composed of greater than 20% (w/w) E100 showed a large increase in the release rate of fluorescent-labeled proteins upon immersion in an acidic environment, and showed negligible release thereafter. The release of FITC-albumin from particles containing 5% (w/w) or less E100 did not appear to be affected by pH. The suspension of particles did not become clear in pH 5, and centrifugation yielded a dense pellet with a color reflecting the fluorescent label that was encapsulated.

The following controls were performed to verify that the increased fluorescent counts seen with acidification were due to the release of the proteins of interest and not of E100 from the particles at pH 5:1) aqueous solutions of E100 in 100 mM sodium acetate pH 5, at concentrations as high as 10 mg/ml, did not cause fluorescence above baseline, 2) when blank (no labeled proteins) 80% (w/w) E100 particles were placed in an acidic environment and then centrifuged, the supernatants did not contain increased fluorescence over baseline.

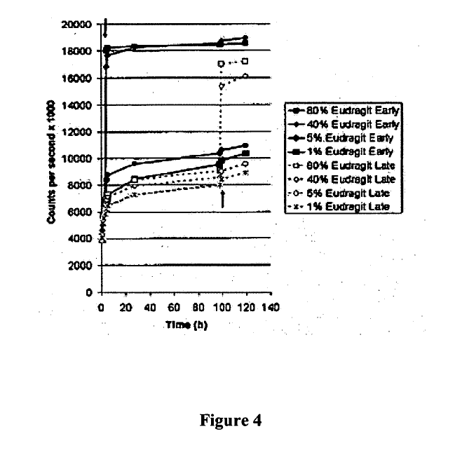

The capacity to release FITC-albumin in response to pH changes was retained for at least 390 hours (16.25 days) after immersion into phosphate-buffered saline (FIG. 3). The capacity for prolonged release and pH triggering was also seen in particles loaded with Rho-lactalbumin (FIG. 4). A larger burst release was noted with Rho-lactalbumin than with FITC-albumin.

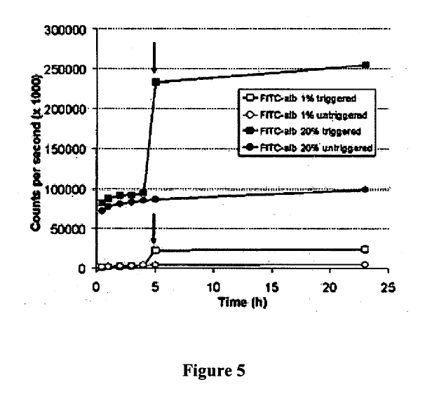

The protein loading in the particles could be increased greatly. We produced particles that contained 1%, 10% or 20% (w/w) FITC-albumin or Rho-lactalbumin and 20% (w/w) E100. These particles had release characteristics similar to those with 0.2% (w/w) protein content, except that they had a large initial burst release (FIG. 5). They displayed a marked release of FITC-albumin upon exposure to pH 5, but retained the coloration of their fluorescent label after pH-triggering, albeit to a much diminished degree.

In Vivo Studies

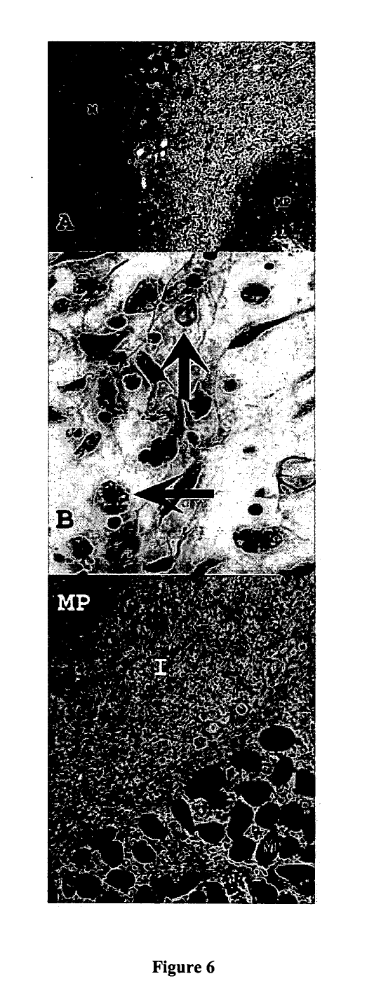

To verify the potential of these particles to attract phagocytic (immune) cells, and to assess their biocompatibility, six animals were injected at the sciatic nerve with 20% (w/w) E100 particles containing 0.2% (w/w) albumin. There was no evidence of self-mutilation at any time after injection in any animal, and the neurobehavioral exam of all animals was normal, with no difference between the injected and contralateral extremities. On dissection one (n=2) and four (n=4) days after injection, well-demarcated pockets of particles were noted at the site of injection. The tissues appeared slightly edematous in the immediate vicinity of those pockets. On hematoxylin-eosin stained sections of tissues harvested from those animals, there was evidence of acute inflammation with neutrophils and macrophages (FIG. 6A), many of which appeared to be laden with particles (FIG. 6B). Inflammation was restricted to the immediate vicinity of the particles, with some infiltration of the adjoining muscle tissue. There was some interstitial edema in the muscle cell layers that were directly adjacent to the area of inflammation, but the myocytes themselves appeared intact (FIG. 6C). Similarly, histological examination of the sites of subcutaneous injections revealed acute inflammation with neutrophils and macrophages. The inflammatory reaction was restricted to the loose connective tissue at the site of injection.

Discussion

The formulations described above provided pH-triggered release of macromolecules at pH 5 across a range of loadings of E100 greater than 20% (w/w). The ability to trigger was not impaired by high protein loadings.

Another benefit of the E100 was that it extended the duration of release of the proteins examined from less than two hours (in particles that did not contain E100) to more than sixteen days (the last time point examined). The capacity to trigger was also maintained during that period. These features may be useful in vivo since the arrival of phagocytic cells to a given site (e.g., subcutaneous depot) often accrues over many days, and we observed that particles were still present in the tissue four days after injection.

In selecting this particle type as a candidate delivery system for intracellular drug delivery, it was apparent that inducing inflammation so as attract immune cells to the site of injection would be a crucial element in determining their effectiveness. Our results supported that assumption. The acute inflammatory reaction to these particles is consistent with the pattern that is seen at this time point in reaction to foreign material, and is similar to what has been described with injected microparticles, including biocompatible microspheres composed of poly(lactic-co-glycolic) acid and lipid-protein-sugar particles similar to the particles described in this report (D. S. Kohane, M. Lipp, R. Kinney, D. Anthony, N. Lotan, and R. Langer. Biocompatibility of lipid-protein-sugar particles containing bupivacaine in the epineurium. J. Biomed. Mat. Res. 59: 450-459 (2002); J. M. Anderson. In vivo biocompatibility of implantable delivery systems and biomaterials. Eur. J. Pharm. Biopharm. 40: 1-8 (1994); J. Castillo, J. Curley, J. Hotz, M. Uezono, J. Tigner, M. Chasin, R. Wilder, R. Langer, and C. Berde. Glucocorticoids prolong rat sciatic nerve blockade in vivo from bupivacaine microspheres. Anesthesiology 85: 1157-66 (1996); each of which is incorporated herein by reference). The presence of macrophages that appeared to be laden with particles suggests that these particles can be taken up by phagocytosis.

E100 is commonly used for enteric coating or flavor-masking of pharmaceutical preparations, but is it not biodegradable and its fate when delivered parenterally is not known (Rohm USA, personal communication). For this reason, we chose to perform injections into a location that included many tissue types, so as to be able to better assess biocompatibility. The tissue injury was mild, and did not extend far outside of the pockets of particles. The fact that there was not evidence of animal distress, self-mutilation, or neurological deficit when the particles were injected at the epineurium (immediately outside the nerve sheath) is also reassuring.

These particles were produced by spray-drying. One advantageous property of that process is that it allows potentially high loadings of the excipients or active molecules of choice. As seen here, particles could be made of 1% to 80% (w/w) E100. Similarly, we achieved 20% (w/w) loading of albumin, and loadings in excess of 60% are easily feasible (data not shown); we have previously described particles that were 36% (w/w) albumin (D. S. Kohane, M. Lipp, R. Kinney, N. Lotan, and R. Langer. Sciatic nerve blockade with lipid-protein-sugar particles containing bupivacaine. Pharm. Res. 17: 1243-1249 (2000); incorporated herein by reference). In principle, such high loadings of DNA could also be possible; we have produced particles that are 4% (w/w) DNA (unpublished observation), but did not attempt higher loadings due to the prohibitive cost. The ability to produce particles with very high loadings of macromolecules is not shared by some more conventional methods of encapsulation into polymeric microspheres (G. Jiang, B. C. Thanoo, and P. P. DeLuca. Effect of osmotic pressure in the solvent extraction phase on BSA release profile from PLGA microspheres. Pharm Dev Technol 7: 391-9 (2002); incorporated herein by reference). Another appealing aspect of this production method is the flexibility it affords in terms of potential excipients, active agents (drugs), and adjuvants. While this report focused on E100 as a model pH-sensitive material, the technique presented here could in principle be applied to any materials with similar properties, such as recently described biopolymers that are both pH-sensitive and biodegradable (D. M. Lynn, D. G. Anderson, D. Putnam, and R. Langer. Accelerated discovery of synthetic transfection vectors: parallel synthesis and screening of a degradable polymer library. J Am Chem Soc 123: 8155-6 (2001); incorporated herein by reference). Another appeal of the spray-drying process is that it is easily amenable to scaling up.

Particles of this type may be useful for stimulating mucosal immunity, particularly in the airway. The lack of effective mucosal antigen delivery is believed to be a major obstacle in the targeting of vaccines to such sites (H. Chen. Recent advances in mucosal vaccine development. J Control Release 67: 117-28 (2000); A. W. Cripps, J. M. Kyd, and A. R. Foxwell. Vaccines and mucosal immunisation. Vaccine 19: 2513-5 (2001); each of which is incorporated herein by reference). Since the immune response is generally strongest at the site of vaccine delivery (L. Stevceva, A. G. Abimiku, and G. Franchini. Targeting the mucosa: genetically engineered vaccines and mucosal immune responses. Genes Immun 1: 308-15 (2000); incorporated herein by reference), it may be advantageous for induction of mucosal immunity in the tracheobronchial tree to be able to deposit the antigen or DNA of interest in the airway. The particle described here could be modified so as that their aerodynamic properties are suitable for inhalational delivery (D. S. Kohane, M. Lipp, R. Kinney, N. Lotan, and R. Langer. Sciatic nerve blockade with lipid-protein-sugar particles containing bupivacaine. Pharm. Res. 17: 1243-1249 (2000); A. Ben-Jebria, D. Chen, M. L. Eskew, R. Vanbever, R. Langer, and D. A. Edwards. Large porous particles for sustained protection from carbachol-induced bronchoconstriction in guinea pigs. Pharm. Res. 16: 555-561 (1999); each of which is incorporated herein by reference); they are already of an appropriate size for that purpose, and as we have seen their density is readily lowered by changing the excipients.

### Example 3

### pH-Triggered Microparticles for Peptide Vaccination

Introduction

Optimizing the CTL response to vaccines is essential to improve the immunotherapy of cancer, and viral diseases (Raychaudhuri, S., and K. L. Rock. 1998. Fully mobilizing host defense: building better vaccines. Nat. Biotechnol. 16:1025; incorporated herein by reference). CD8⁺ T cells will only respond to vaccine antigens in vivo if the epitopes contained in the vaccine are presented in the context of MHC I by specialized antigen presenting cells (APCs), such as dendritic cells (DCs). The amount of antigen presented at the time of initial encounter between T cell and the APC is a critical factor that dictates the strength of T cell stimulation. Increasing the epitope density decreases the threshold for activation of naive T cells and increases the size of the primary T cell response (Gett, A. V., F. Sallusto, A. Lanzavecchia, and J. Geginat. 2003. T cell fitness determined by signal strength. Nat. Immunol. 4:355; Wherry, E. J., K. A. Puorro, A. Porgador, and L. C. Eisenlohr. 1999. The induction of virus-specific CTL as a function of increasing epitope expression: responses rise steadily until excessively high levels of epitope are attained. J. Immunol. 163:3735; Kaech, S. M., and R. Ahmed. 2001. Memory CD8⁺ T cell differentiation: initial antigen encounter triggers a developmental program in naïve cells. Nat. Immunol. 2:415; Bullock, T. N., D. W. Mullins, and V. H. Engelhard. 2003. Antigen density presented by dendritic cells in vivo differentially affects the number and avidity of primary, memory, and recall CD8⁺ T cells. J. Immunol. 170:1822; each of which is incorporated herein by reference). APCs can present soluble exogenous antigens such as those given in vaccines to CD8⁺ cells by what is known as cross-presentation (Bevan, M. J. 1976. Cross-priming for a secondary cytotoxic response to minor H antigens with H-2 congenic cells which do not cross-react in the cytotoxic assay. J. Exp. Med. 143:1283; Heath, W. R., and F. R. Carbone. 2001. Cross-presentation, dendritic cells, tolerance and immunity. Annu. Rev. Immunol. 19:47; each of which is incorporated herein by reference), but the process is inefficient (Kovacsovics-Bankowski, M., K. Clark, B. Benacerraf, and K. L. Rock. 1993. Efficient major histocompatibility complex class I presentation of exogenous antigen upon phagocytosis by macrophages. Proc. Natl. Acad. Sci. USA 90:4942; Kovacsovics-Bankowski, M., and K. L. Rock. 1995. A phagosome-to-cytosol pathway for exogenous antigens presented on MHC class I molecules. Science 267:243; Shen, Z., G. Reznikoff, G. Dranoff, and K. L. Rock. 1997. Closed dendritic cells can present exogenous antigens on both MHC class I and class II molecules. J. Immunol. 158:2723; each of which is incorporated herein by reference). Encapsulation of protein antigen (Ag) in a particulate form that can be phagocytosed by APCs markedly enhances Ag presentation and the resulting CTL response (Kovacsovics-Bankowski, M., K. Clark, B. Benacerraf, and K. L. Rock. 1993. Efficient major histocompatibility complex class I presentation of exogenous antigen upon phagocytosis by macrophages. Proc. Natl. Acad. Sci. USA 90:4942; Kovacsovics-Bankowski, M., and K. L. Rock. 1995. A phagosome-to-cytosol pathway for exogenous antigens presented on MHC class I molecules. Science 267:243; Shen, Z., G. Reznikoff, G. Dranoff, and K. L. Rock. 1997. Closed dendritic cells can present exogenous antigens on both MHC class I and class II molecules. J. Immunol. 158:2723; each of which is incorporated herein by reference). Recent work has identified specialized cellular mechanisms by which antigens engulfed in acidic phagosomes directly enter the MHC I pathway (Rodriguez, A., A. Regnault, M. Kleijmeer, P. Ricciardi-Castagnoli, and S. Amigorena. 1999. Selective transport of internalized antigens to the cytosol for MHC class I presentation in dendritic cells. Nat. Cell. Biol. 1:362; Guermonprez, P., L. Saveanu, M. Kleijmeer, J. Davoust, P. Van Endert, and S. Amigorena. 2003. ER-phagosome fusion defines an MHC class I cross-presentation compartment in dendritic cells. Nature 425:397; Houde, M., S. Bertholet, E. Gagnon, S. Brunet, G. Goyette, A. Laplante, M. F. Princiotta, P. Thibault, D. Sacks, and M. Desjardins. 2003. Phagosomes are competent organelles for antigen cross-presentation. Nature 425:402; each of which is incorporated herein by reference). Targeting vaccine antigens to the phagosome by encapsulating them in microparticles therefore represents a way to improve the presentation of vaccine antigens to CD8⁺ cells, thereby enhancing the CTL response to peptide/protein vaccines.

Controlled release technology has been used by many investigators to encapsulate vaccine antigens for delivery to APCs. Microparticles made from polymeric biomaterials such as the α-hydroxy acids, including poly(lactic-coglycolic) acid, have been used extensively (Hanes, J., J. L. Cleland, and R. Langer. 1997. New advances in microsphere-based single-dose vaccines. Adv. Drug Delivery Rev. 28:97; Nixon, D. F., C. Hioe, P. D. Chen, Z. Bian, P. Kuebler, M. L. Li, H. Qiu, X. M. Li, M. Singh, J. Richardson, et al. 1996. Synthetic peptides entrapped in microparticles can elicit cytotoxic T cell activity. Vaccine 14:1523; Moore, A., P. McGuirk, S. Adams, W. C. Jones, J. P. McGee, D. T. O'Hagan, and K. H. Mills. 1995. Immunization with a soluble recombinant HIV protein entrapped in biodegradable microparticles induces HIV-specific CD8⁺ cytotoxic T lymphocytes and CD4⁺ Th1 cells. Vaccine 13:1741; each of which is incorporated herein by reference). However, one problem with poly(lactic-coglycolic) acid microparticles is their slow degradation. Even when those particles are small, and modified to degrade relatively rapidly, they can still be found in situ weeks after injection (Kohane, D. S., M. Lipp, R. Kinney, D. Anthony, N. Lotan, and R. Langer. 2002. Biocompatibility of lipid-protein-sugar particles containing bupivacaine in the epineurium. J. Biomed. Mater. Res. 59:450; incorporated herein by reference). This slow degradation may lead to suboptimal intracellular delivery of the antigenic payload. One method of overcoming this problem is to make the microparticles from excipients that are pH sensitive (Lynn, D. M., M. Amiji, and R. Langer. 2001. pH-responsive polymer microspheres: rapid release of encapsulated material within the range of intracellular pH. Angew. Chem. Int. Ed. 40:1707; Kohane, D. S., D. G. Anderson, C. Yu, and R. Langer. 2003. pH-triggered release of macromolecules from spray-dried polymethacrylate microparticles. Pharm. Res. 20:1533; each of which is incorporated herein by reference). Such particles remain intact at the physiological pH of the extracellular fluid, but once taken up by APCs, could disintegrate in the acidic environment of the phagosome (Hackam, D. J., O. D. Rotstein, W. J. Zhang, N. Demaurex, M. Woodside, O. Tsai, and S. Grinstein. 1997. Regulation of phagosomal acidification: differential targeting of Na⁺/H⁺ exchangers, Na⁺/K⁺-ATPases, and vacuolar-type H⁺-atpases. J. Biol. Chem. 272:29810; incorporated herein by reference). The rapid release of the particles' contents directly into an organelle rich in the machinery of antigen presentation (Guermonprez, P., L. Saveanu, M. Kleijmeer, J. Davoust, P. Van Endert, and S. Amigorena. 2003. ER-phagosome fusion defines an MHC class I cross-presentation compartment in dendritic cells. Nature 425:397; Houde, M., S. Bertholet, E. Gagnon, S. Brunet, G. Goyette, A. Laplante, M. F. Princiotta, P. Thibault, D. Sacks, and M. Desjardins. 2003. Phagosomes are competent organelles for antigen cross-presentation. Nature 425:402; Ackerman, A. L., C. Kyritsis, R. Tampe, and P. Cresswell. 2003. Early phagosomes in dendritic cells form a cellular compartment sufficient for cross presentation of exogenous antigens. Proc. Natl. Acad. Sci. USA 100:12889; each of which is incorporated herein by reference) should facilitate loading onto MHC I.

We have described the generation of phospholipid-based microparticles that have been rendered pH triggerable by incorporation of a polymethacrylate (Eudragit E100 (E100)) as a model pH-sensitive material (Kohane, D. S., D. G. Anderson, C. Yu, and R. Langer. 2003. pH-triggered release of macromolecules from spray-dried polymethacrylate microparticles. Pharm. Res. 20:1533; each of which is incorporated herein by reference). These particles have properties that are potentially attractive for vaccine delivery. They are typically 2-6 μm in diameter, so they can only be taken up by cells that are capable of phagocytosis (Tabata, Y., and Y. Ikada. 1990. Phagocytosis of polymer microspheres by macrophages. Adv. Polymer. Sci. 94:107; incorporated herein by reference). They are composed of a variety of inert excipients, typically phospholipids, sugars, proteins, and other macromolecules, and the molecule (drug) of interest. Excipients can be selected that are appropriate for the milieu to which the microparticles will be delivered, thus optimizing biocompatibility (Kohane, D. S., M. Lipp, R. Kinney, D. Anthony, N. Lotan, and R. Langer. 2002. Biocompatibility of lipid-protein-sugar particles containing bupivacaine in the epineurium. J. Biomed. Mater. Res. 59:450; incorporated herein by reference).

Furthermore, the process by which the micro-particles are produced, spray drying, allows relatively high loadings of molecules of interest; for example, they can be made to contain 36% (w/w) albumin (Kohane, D. S., M. Lipp, R. Kinney, N. Lotan, and R. Langer. 2000. Sciatic nerve blockade with lipid-protein-sugar particles containing bupivacaine. Pharm. Res. 17:1243; incorporated herein by reference). Injection of microparticles of this type attracts immune cells to the site of injection as part of an acute inflammatory response that could potentiate the T cell response to vaccination (Kohane, D. S., D. G. Anderson, C. Yu, and R. Langer. 2003. pH-triggered release of macromolecules from spray-dried polymethacrylate microparticles. Pharm. Res. 20:1533; incorporated herein by reference).

In this study, we describe the use of pH-sensitive microparticles composed of a phospholipid, dipalmitoyl-phosphatidylcholine (DPPC), and the pH-sensitive material E100 (Kohane, D. S., D. G. Anderson, C. Yu, and R. Langer. 2003. pH-triggered release of macromolecules from spray-dried polymethacrylate microparticles. Pharm. Res. 20:1533; incorporated herein by reference) as delivery vehicles for peptide antigens. We show pH-dependent release of an MHC I-restricted peptide epitope from influenza A matrix protein, and demonstrate efficient delivery of this epitope to human DCs. Encapsulation of the antigen in pH-triggered particles markedly enhances presentation of the peptide to CD8⁺ T cells in vitro compared with pH-insensitive particles, and allows priming of CTL responses to the epitope in human HLA-A*0201 transgenic mice.

Materials and Methods

Peptides and Other Reagents

The 9-aa peptide M58 with the sequence GILGFVFTL was obtained from New England Peptide (Fitchburg, Mass.) with or without conjugation to the fluorophore AMC (AMC-M58). DPPC was obtained from Avanti Polar Lipids (Alabaster, Ala.). E100, poly(butyl methacrylate-co-(2-dimethylaminoethyl) methacrylate-co-methyl methacrylate)=1:2:1, was a gift of Rohm and Haas (Philadelphia, Pa.). FITC-labeled albumin, rhodamine isothiocyanate (p)-labeled lactalbumin, and poly-HEME were obtained from Sigma-Aldrich (St. Louis, Mo.). Polyinosinic:polycytidylic acid (poly(I:C)) was obtained from Sigma-Aldrich.

Production and Characterization of Microparticles

Particles containing FITC-albumin or p-lactalbumin were made as follows. One hundred milligrams of E100 or poly-HEME, and 400 mg of DPPC were dissolved in 87.5 ml of ethanol. One milligram of either labeled protein in 37.5 ml of water was added dropwise to the ethanol solution. The mixture was then fed into a Buchi 190 bench-top spray drier at the following settings: air flow, 600 NI/h; inlet temperature, 110° C.; aspiration, −18 mbar; solvent flow rate, 12 ml/min. At these settings, the outlet temperature was ˜40° C.

Particles containing M58 peptide or AMC-M58 peptide were produced, as follows. M58 peptide was dissolved in acetonitrile:ethanol:water 20:56:24 with 0.1% trifluoroacetic acid, to a peptide concentration of 1 mg/ml. One hundred milligrams of E100 or poly-HEME, and 400 mg of DPPC were dissolved in ethanol, and water was added dropwise until the final volume was 125 ml minus the volume of M58 solution to be added. The pH of the solution was measured as the M58 solution was added. The pH was then adjusted back to initial value with NaOH. The mixture was spray dried, as above.

Particle Size and Shape Determination

The size of particles was determined with a Coulter counter (Coulter Electronics, Luton, U.K.) using a 30 μm orifice. The morphologies of selected particles were assessed by scanning electron microscopy using an AMR-1000 at 10 kV using a gold-palladium conductive coating.

In Vitro Release of FITC-Labeled Albumin

Five-milligram aliquots of particles were suspended in 1 ml of PBS, pH 7.4, and incubated at 37° C. At predetermined time points, the sample was centrifuged (8000 rpm for 4 min), and the supernatant was removed. Samples were resuspended into PBS or 1.5 h or 4 days after initial suspension, into 100 mM sodium acetate, pH 5. Once suspended in sodium acetate, samples were kept in that solution. The fluorescence in the supernatant was quantitated with a PTI system (Photon Technology International, Lawrenceville, N.J.) at the following wavelengths (excitation and emission, respectively): FITC-albumin, 485, 515; AMC-M58, 350,447.

Donors and Cell Lines

Leukapharesis products were obtained from healthy blood donors with appropriate consent from the Dana-Farber/Harvard Cancer Center Institutional Review Board (Boston, Mass.). PBMC were purified by Ficoll density centrifugation and cryopreserved. Immature DCs were generated from plastic-adherent monocytes by culture with IL-4 and GM-CSF, as described (Von Bergwelt-Baildon, M. S., R. H. Vonderheide, B. Maecker, N. Hirano, K. S. Anderson, M. O. Butler, Z. Xia, W. Y. Zeng, K. W. Wucherpfennig, L. M. Nadler, and J. L. Schultze. 2002. Human primary and memory cytotoxic T lymphocyte responses are efficiently induced by means of CD40-activated B cells as antigen-presenting cells: potential for clinical application. Blood 99:3319; incorporated herein by reference).

Human T cell lines specific for M58 peptide were generated, as described (Von Bergwelt-Baildon, M. S., R. H. Vonderheide, B. Maecker, N. Hirano, K. S. Anderson, M. O. Butler, Z. Xia, W. Y. Zeng, K. W. Wucherpfennig, L. M. Nadler, and J. L. Schultze. 2002. Human primary and memory cytotoxic T lymphocyte responses are efficiently induced by means of CD40-activated B cells as antigen-presenting cells: potential for clinical application. Blood 99:3319; incorporated herein by reference). Clones were generated by plating T cells from lines with peptide-specific cytotoxic activity at 0.3 cells/well with irradiated EBV-lymphoblastoid lines and allogeneic PBMC together with soluble CD3 (OKT3) and IL-2 (100 U/ml); Chiron, Emeryville, Calif.). Wells with growing clusters were expanded by restimulating with the same combination of allogeneic feeder cells, CD3 Ab, and IL-2 before being screened for cytotoxic activity. The clone used for experiments was CD8⁺, and stained strongly with an HLA-A*0201-peptide tetramer containing M58 peptide.

HLA-A*0201 Transgenic Mice and Immunization Procedures

HHD mice express a chimeric human (α1 and α2 chains) and murine (α3 chain) HLA-A*0201 chain covalently linked to the human β2-microglobulin L chain. The murine MHC I molecule H-2 Db has been deleted (Firat, H., F. Garcia-Pons, S. Tourdot, S. Pascolo, A. Scardino, Z. Garcia, M. L. Michel, R. W. Jack, G. Jung, K. Kosmatopoulos, et al. 1999. H-2 class I knockout, HLA-A2.1-transgenic mice: a versatile animal model for preclinical evaluation of antitumor immunotherapeutic strategies. Eur. J. Immunol 29:3112; incorporated herein by reference). HHD mice were injected s.c. at the base of the tail with 100 μg of M58 peptide or the corresponding amount of peptide encapsulated in microparticles. No other adjuvant was given. After 7 days, splenocytes from primed HHD mice were harvested and restimulated with peptide-loaded HHD lymphoblasts, as previously described (Firat, H., F. Garcia-Pons, S. Tourdot, S. Pascolo, A. Scardino, Z. Garcia, M. L. Michel, R. W. Jack, G. Jung, K. Kosmatopoulos, et al. 1999. H-2 class I knockout, HLA-A2.1-transgenic mice: a versatile animal model for preclinical evaluation of antitumor immunotherapeutic strategies. Eur. J. Immunol 29:3112; incorporated herein by reference). Six days later, cultured cells were tested for cytotoxic activity in a 4-h ⁵¹Cr release assay, using as targets either HHD-transfected TAP⁻ RMA-S cells loaded with M58 or negative control RT Pol 476 (SYNT:EM, Nimes, France) peptides (10 μg/ml).

ELISPOT Analysis

ImmunoSpot plates (Cellular Technology, Cleveland, Ohio) were prepared by precoating with 5 μg/ml anti-IFN-γ AB (Mabtech, Nacka, Sweden) overnight at 37° C. DCs were loaded overnight with particles containing M58 peptide or with free peptide, harvested, washed, and plated with T cells in varying ratios, and incubated at 37° C. for 18 hours. After washing, wells were developed, according to the manufacturer's recommendations, and the spots were visualized with a 5-bromo-4-chloro-3-indolyl-phosphate and NBT color development substrate (Bio-Rad, Hercules, Calif.). An Immunospot Analyzer (Cellular Technology) was used to record and analyze images of wells from developed plates.

Flow Cytometry and Immunofluorescence Microscopy

DCs or PBMCs that had been exposed to varying concentrations of microparticles, FITC-albumin, or poly(I:C) (10 ng/ml) were washed and stained with Abs for relevant surface markers (Beckman Coulter, Gainsville, Fla.), or with annexin-V (R&D Systems, Minneapolis, Minn.) using FITC, PE, or PE-Cy7 as fluorophores. Quantification of uptake of FITC-albumin by different cell populations was determined using flow cytometry, and exclusion of unincorporated particles was done by setting gates on plots of relevant lineage markers vs right-angle light scatter.

For immunofluorescence microscopy, DCs were exposed to rhodamine-albumin particles for 1-16 h (5 μg/ml), fixed with 1% formaldehyde, and permeabilized with Triton X-100 (0.1%). DCs were then stained with Alexa Fluor 488 phalloidin and, in some experiments, 4′,6-diamidino-2-phenylindole, dihydrochloride (both from Molecular Probes, Eugene, Oreg.), according to manufacturer's instructions. Fluorescence microscopy images were acquired using a Zeiss (Oberkochen, Germany) Axiovert microscope, and deconvolution analysis was performed with Openlab Deconvolution Software (Improvision, Lexington, Mass.).

Time-Lapse Video Microscopy

DCs were harvested and allowed to adhere to 1.5-cm tissue culture plates (Corning-Costar, Acton, Mass.) overnight, and placed in a chamber connected to a source of 10% CO₂ balanced air. The chamber was placed on a 37° C. heating stage. Particles were added to the medium overlying the DCs and allowed to settle for 10 min before the initiation of recording. Images were recorded using an Olympus IX70 microscope connected to a digital camera (Digital Video Camera Company, Austin, Tex.). Images of selected fields in differential interference contrast were captures with an interval of 30 s over a period of 1 h using QED software with a time-lapse module (QED Imaging, Pittsburgh, Pa.).

Results

Generation of pH-Triggered Microparticles Containing M58 Peptide

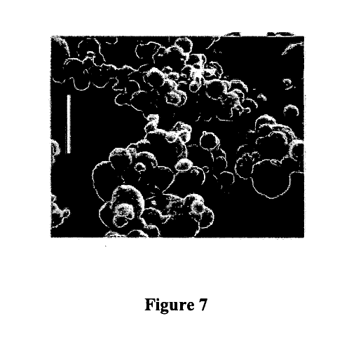

Particles containing 0.2% (w/w) FITC-albumin, 0.2% (w/w) M58 peptide (with and without AMC-M58 peptide), or 20% (w/w) p-lactalbumin were generated, as described in Materials and Methods, all containing 20% (w/w) E100. In addition, 20% (w/w) poly-HEME particles were produced containing 0.2% (w/w) FITC-albumin, or 20% (w/w) p-lactalbumin. The manufacture process produced a fine powder that was yellow with FITC-albumin, white with M58 or AMC-M58, and bright pink with p-lactalbumin. The powder yield was 20-40% of the total solute. Particles were generally spheroidal (FIG. 7). The median volume-weighted diameters of all particles were in the range of 4-6 μm.

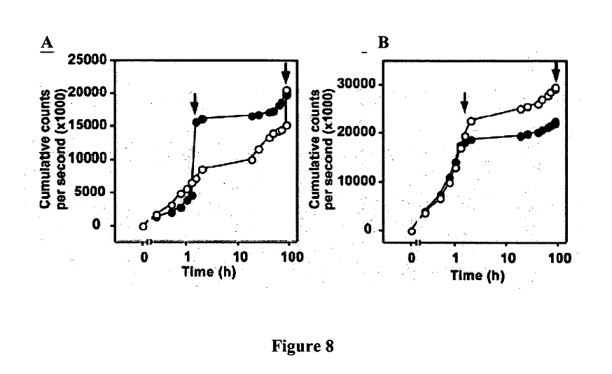

The kinetics of peptide release were studied in vitro (FIGS. 8A and 8B). At pH 7.4, release of M58 peptide occurred very slowly from both E100- and poly-HEME-based microparticles when the suspending medium was changed to sodium acetate, pH 5 (FIG. 8A). In E100 particles, this burst could still be triggered after 4 days in suspension at pH 7.4. Poly-HEME microparticles did not show an increase in peptide release upon immersion in acidic pH, neither shortly after immersion in PBS nor 4 days later (FIG. 8B). FITC-albumin containing E100 microparticles were similarly pH responsive to acidic environments (data not shown) (see also Kohane, D. S., D. G. Anderson, C. Yu, and R. Langer. 2003. pH-triggered release of macromolecules from spray-dried polymethacrylate microparticles. Pharm. Res. 20:1533; incorporated herein by reference).

Uptake of Microparticles by DCs and Monocytes

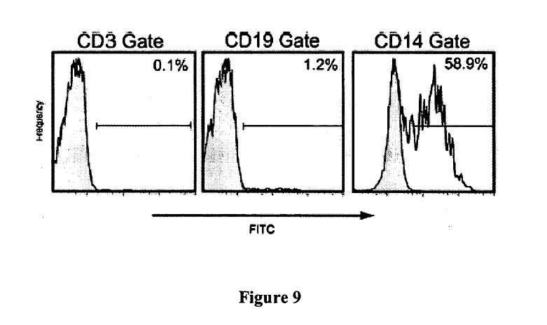

To assess the efficiency of particle uptake by different cell populations, PBMCs were cultured overnight with microparticles containing FITC-albumin (FIG. 9), and the relative FITC-fluorescence in T cells, B cells, and monocytes was determined by flow cytometry. The majority of monocytes (CD14⁺ large cells) were fluorescently labeled with FITC. In contrast, almost none of the T or B cells were FITC labeled.

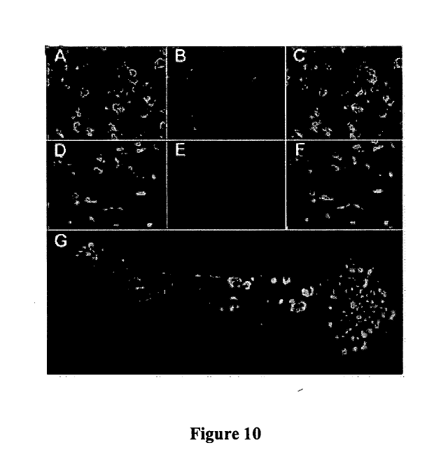

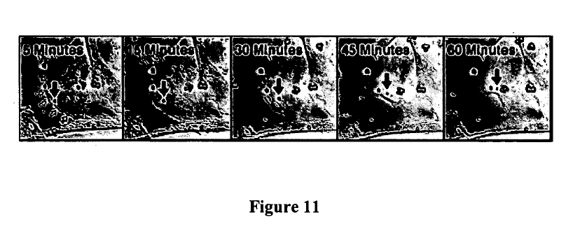

Microparticles (0.2% (w/w) FITC-albumin, 20% (w/w) E100) were also efficiently engulfed by immature DCs (FIG. 10). Immature, monocyte-derived DCs were prepared using established methods, and their interaction with 20% (w/w) p-lactalbumin, 20% (w/w) E100 microparticles was studied by fluorescence microscopy (FIG. 10). DCs were cultured with microparticles for 1-2 h, labeled with a fluorescent phalloidin to delineate the actin cytoskeleton, and then washed thoroughly to remove nonadherent or extracellular particles. After incubation at 37° C., most DCs were associated with one or more microparticles (FIG. 10A-10C), and deconvolution analysis of acquired images confirmed that the particles were localized intracellularly, clustered in the perinuclear region of the cells (FIG. 10G). DCs were also imaged at later time points, and engulfed particles were still visible in cells 48-72 h after loading (data not shown). However, if DCs were incubated at 4° C. (FIG. 10D-10F), no particles were visible in association with the cells, suggesting that the uptake of particles was an energy-dependent process. Time-lapse video microscopy was used to visualize the dynamics of this interaction at 37° C. Representative images from a 1-h time course are shown in FIG. 11. Microparticles could be identified as highly refractile objects of subcellular size that were rapidly withdrawn toward the cell body and were engulfed over a period of 15-45 min. These data show that pH-triggered microparticles are preferentially, avidly, and rapidly phagocytosed by professional APCs.

DC Viability, Phenotype, and Function After Particle Loading

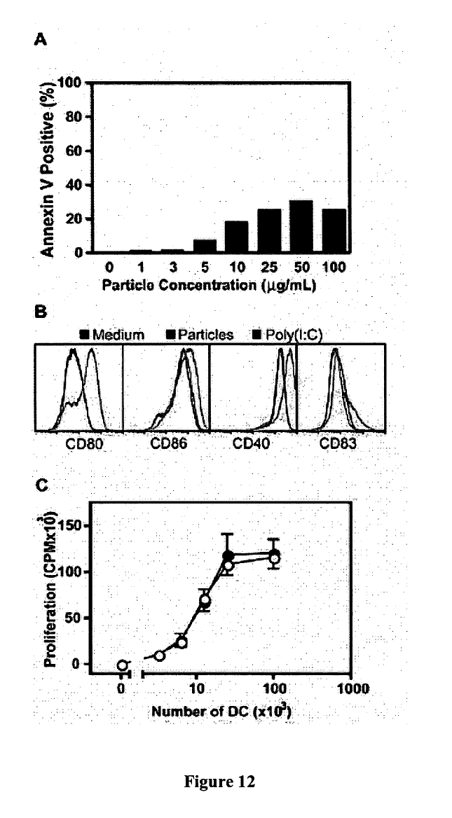

A theoretical concern about the uptake of microparticles by DCs is that it may cause cytotoxicity or disrupt DC function. We therefore assessed DC viability, maturation, and function following coculture with microparticles. Immature DCs were cocultured with a range of concentrations of 0.2% (w/w) FITC-albumin, 20% (w/w) E100 microparticles overnight (FIG. 12A), and the degree of cell death was measured by annexin-V binding. At concentrations of microparticles lower than 10 μg/ml, <10% of cells were apoptotic (annexin-V positive). At concentrations greater than 10 μg/ml, there was a modest increase in cell death to 20-30%. However, concentrations of microparticles that increased apoptosis in DCs were in excess of those necessary for efficient loading (see below). To assess the microparticles' effect on DC maturation, we measured the expression of CD80, CD86, CD40, and CD83 in DCs cultured with 5 μg/ml 0.2% (w/w) FITC-albumin, 20% (w/w) E100 microparticle (10 μg/ml). The dsRNA complex poly(I:C) was used as a positive control. After 48 h of culture, poly(I:C) induced marked up-regulation of CD80, CD86, and CD40 on the majority of cells, and a subset of cells showed increased expression of CD83 (FIG. 12B). In contrast, the expression levels of these surface markers were unchanged by culture with microparticles, suggesting that they did not influence the maturation state of the DCs (FIG. 12B). We further assessed the effect of microparticle uptake on APC function by measuring the ability of DCs to stimulate allogeneic T cells following incubation with microparticles (0.2% (w/w) FITC-albumin, 20% (w/w) E100) or with a comparable concentration of soluble FITC-albumin as a control. FIG. 12C shows that the degree of T cell proliferation elicited by DCs cocultured with 5 μg/ml those microparticles was identical with that of control DCs. These data suggest that the uptake of microparticles is not toxic to DCs, and perturbs neither their maturation state nor their ability to stimulate T cells.

Uptake of Soluble vs Encapsulated FITC-Albumin

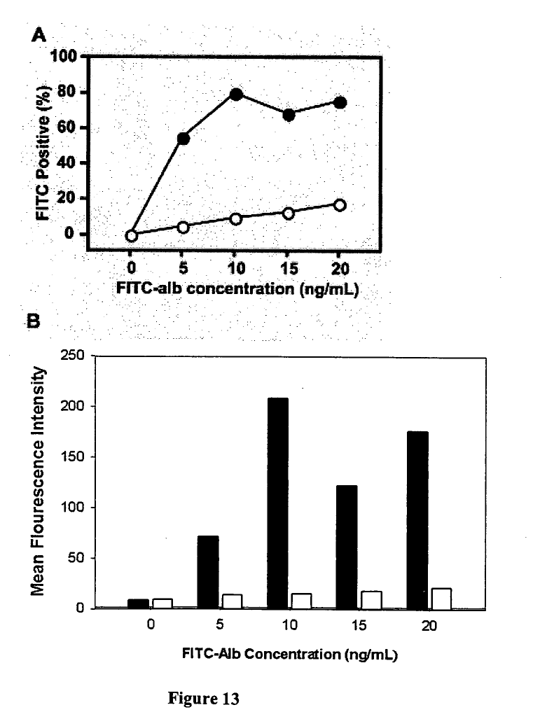

The avid phagocytosis of microparticles by DCs suggested that they would be more effective at delivering a potential antigen to APCs. We therefore compared the ability of encapsulated protein to enter DCs With that of unencapsulated protein. Immature DCs were cultured in the presence of unencapsulated FITC-albumin or of equivalent concentrations of FITC-albumin as 0.2% (w/w) FITC-albumin, 20% (w/w) E100 microparticles overnight. Flow cytometry revealed that even at low particle concentrations (e.g., 5 μg/ml particle, which corresponds to 10 ng/ml encapsulated FITC-albumin), the majority of DCs were labeled with FITC, up to a maximum of ˜80% (FIG. 13A). At all concentrations examined, uptake of FITC-albumin was much higher with encapsulated FITC-albumin than with the corresponding concentration of unencapsulated FITC-albumin measured both by percentage of labeled DCs and the fluorescence intensity (FIG. 13B). Thus, the phagocytosis of microparticles increased the delivery of encapsulated antigen to DCs.

Peptide Ag Presentation by Microparticle-Loaded Human DCs

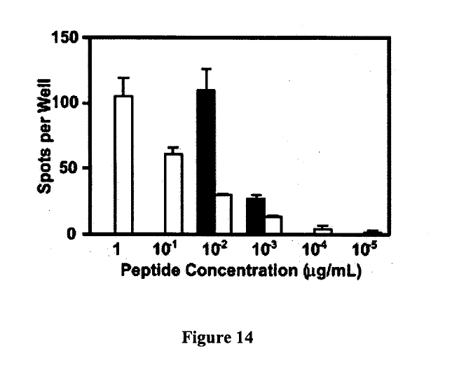

Improved Ag delivery to DCs is a critical component of antigen presentation. However, to elicit CD8⁺ T cell responses, phagocytosed Ag must efficiently enter the MHC I presentation pathway. We tested the effect of encapsulation on the ability of DCs to present a peptide epitope, the immunodominant epitope from influenza A matrix protein, to CD8⁺ T cells. Because wide variations in particle concentrations might influence antigen presentation, we used a fixed concentration of particles and prepared two particle formulations that delivered the peptide concentrations equivalent to 10⁻² μg/ml. or 10⁻³ μg/ml. DCs pulsed with unencapsulated M58 peptide stimulated a peptide-specific HLA-A*0201-restricted T cell clone in a peptide concentration-dependent fashion (FIG. 14). However, at two concentrations (0.2% (w/w) M58, 20% (w/w) E100 microparticles), encapsulated Ag was much more efficient at stimulating a T cell response than the equivalent concentration of soluble peptide. For instance, encapsulated peptide equivalent to a concentration of 10⁻² μg/ml achieved the same T cell response as that achieved by 1 μg/ml free peptide. This suggests that encapsulating a CD8⁺ epitope in pH-triggered microparticles markedly increases the presentation of peptide epitopes on MHC I of DCs.

Role of pH Triggering In Vitro

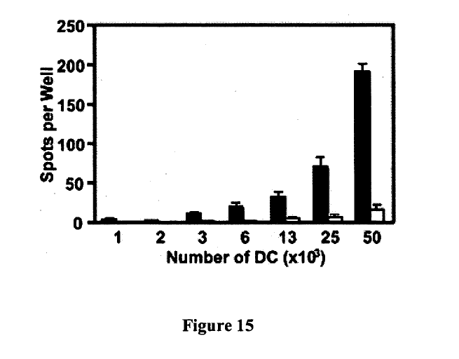

The contribution of pH triggering to this improved Ag presentation was assessed by comparing peptide delivery to DCs by pH-triggered E100 particles and pH-insensitive microparticles prepared in the same matter, except that e100 was replaced by poly-HEME. Both types of particles were taken up by DCs with equivalent efficacy and were equally nontoxic (data not shown). DCs were cultured overnight in medium containing 5 μg/ml microparticles containing 0.2% (w/w) M58 peptide and either 20% (w/w) of E100 or 20% (w/w) of poly-HEME (FIG. 13). Poly-HEME microparticles elicited very little T cell stimulation. In contrast, pH-triggered microparticles elicited T cell stimulation that was markedly greater than that induced by nontriggering microparticles (FIG. 15).

Vaccination Using Encapsulated Peptide Ag

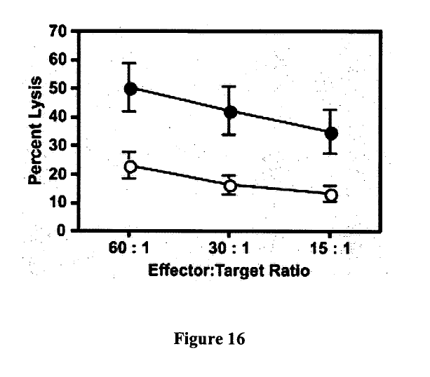

The in vitro results with a CD8⁺ T cell clone suggested that encapsulation of the peptide in pH-triggered microparticles increased presentation of antigen by MHC I markedly. However, for application as part of a vaccine, encapsulated antigen should also be able to stimulate naïve CD8+ T cells. We tested naïve CD8+ T cell priming to the M58 epitope by vaccinating HLA-A*0201 transgenic HHD mice. HLA-A*0201 transgenic mice such as HHD mice have been used extensively in the study of naïve T cell responses to neo-Ags and have an immunodominant response to the M58 epitope from influenza A that is similar to HLA-A*0201-bearing humans (Firat, H., F. Garcia-Pons, S. Tourdot, S. Pascolo, A. Scardino, Z. Garcia, M. L. Michel, R. W. Jack, G. Jung, K. Kosmatopoulos, et al. 1999. H-2 class I knockout, HLA-A2.1-transgenic mice: a versatile animal model for preclinical evaluation of antitumor immunotherapeutic strategies. Eur. J. Immunol 29:3112; Vitiello, A., D. Marchesini, J. Furze, L. A. Sherman, and R. W. Chesnut. 1991. Analysis of the HLA-restricted influenza-specific cytotoxic T lymphocyte response in transgenic mice carrying a chimeric human-mouse class I major histocompatibility complex. J. Exp. Med. 173:1007; Pascolo, S., N. Bervas, J. M. Ure, A. G. Smith, F. A. Lemonnier, and B. Peramau. 1997. HLA-A2.1-restricted education and cytolytic activity of CD8⁺ T lymphocytes from β2 microglobulin (β₂m) HLA-A2.1 monochain transgenic H-2 Db β₂m double knockout mice. J. Exp. Med. 185:2043; Shirai, M., T. Arichi, M. Nishioka, T. Nomura, K. Ikeda, K. Kawanishi, V. H. Engelhard, S. M. Feinstone, and J. A. Berzofsky. 1995. CTL responses of HLA-A2.1-transgenic mice specific for hepatitis C viral peptides predict epitopes for CTL of humans carrying HLA-A2.1. J. Immunol. 154:2733; Wentworth, P. A., A. Vitiello, J. Sidney, E. Keogh, R. W. Chesnut, H. Grey, and A. Sette. 1996. Differences and similarities in the A2.1-restricted cytotoxic T cell repertoire in humans and human leukocyte antigen-transgenic mice. Eur. J. Immunol. 26:97; Graff-Dubois, S., O. Faure, D. A. Gross, P. Alves, A. Scardino, S. Chouaib, F. A. Lemonnier, and K. Kosmatopoulos. 2002. Generation of CTL recognizing an HLA-A*0201-restricted epitope shared by MAGE-A1, -A2, -A3, -A4, -A6, -A10, and -A12 tumor antigens: implication in a broad-spectrum tumor immunotherapy. J. Immunol. 169:575; Francini, G., A. Scardino, K. Kosmatopoulos, F. A. Lemonnier, G. Campoccia, M. Sabatino, D. Pozzessere, R. Petrioli, L. Lozzi, P. Neri, et al. 2002. High-affinity HLA-A(*)02.01 peptides from parathyroid hormone-related protein generate in vitro and in vivo antitumor CTL response without autoimmune side effects. J. Immunol. 169-4840; Plonquet, A., F. Garcia-Pons, E. Fernandez, C. Philippe, J. Marquet, H. Rouard, M. H. Delfau-Larue, K. Kosmatopoulos, F. Lemonnier, J. P. Farcet, at al. 2003. Peptides derived from the onconeural HuD protein can elicit cytotoxic responses in HHD mouse and human. J. Neuroimmunol. 142:93; Alves, P. M., O. Faure, S. Graff-Dubois, D. A. Gross, S. Comet, S. Chouaib, I. Miconnet, F. A. Lemonnier, and K. Kosmatopoulos. 2003. EphA2 as target of anticancer immunotherapy: identification of HLA-A*0201-restricted epitopes. Cancer Res. 63:8476; Faure, O., S. Graff-Dubois, L. Bretaudeau, L. Derre, D. A. Gross, P. M. Alves, S. Comet, M. T. Duffour, S. Chouaib, I. Miconnet, et al. 2004. Inducible Hsp70 as target of anticancer immunotherapy: identification of HLA-A*0201-restricted epitopes. Int. J. Cancer 108:863; Gross, D. A., S. Graff-Dubois, P. Opolon, S. Comet, P. Alves, A. Bennaceur-Griscelli, O. Faure, P. Guillaume, H. Firat, S. Chouaib, et al. 2004. High vaccination efficiency of low-affinity epitopes in antitumor immunotherapy. J. Clin. Invest. 113:425; each of which is incorporated herein by reference). HHD mice (n=5 each group) were vaccinated once with M58 peptide in saline or encapsulated in microparticles. Results shown in FIG. 14 show that encapsulation of M58 allowed the priming of peptide-specific CTL with robust cytotoxic activity significantly greater than that induced by injection of peptide alone. For instance, at an E:T ratio of 30:1, lysis with T cells from particle-vaccinated HHD mice was 42 vs 16% for mice immunized with soluble peptide. Lysis of targets pulsed with an irrelevant peptide by T cells from either group of mice was on average 1.5% and always <8% (FIG. 16).

Discussion

In this study, we show that encapsulation in pH-triggered microparticles markedly increases the delivery of a peptide Ag to the MHC I pathway of human DCs and improves T cell stimulation in vitro and in vivo.

Microparticles composed of 20% (w/w) E100 can encapsulate peptide and protein Ags, and provide both sustained and pH-triggered release of the peptide in vitro. The effect of greatly prolonging the release of peptide from DPPC-based particles at physiological pH (Kohane, D. S., D. G. Anderson, C. Yu, and R. Langer. 2003. pH-triggered release of macromolecules from spray-dried polymethacrylate microparticles. Pharm. Res. 20:1533; each of which is incorporated herein by reference) is important in that it may take days for all injected particles to be phagocytosed by the cell of interest (Kohane, D. S., M. Lipp, R. Kinney, D. Anthony, N. Lotan, and R. Langer. 2002. Biocompatibility of lipid-protein-sugar particles containing bupivacaine in the epineurium. J. Biomed. Mater. Res. 59:450; each of which is incorporated herein by reference). Because the cell surface-active properties of the polymethacrylates used in this study (E100 and poly-HEME) were not known, but both could potentially have effects on phagocytosis, it was important to document that the particles whether biologically effective or not, actually entered the cells. pH-triggered microparticles were phagocytosed by DCs efficiently and rapidly. Deconvolution microscopy confirmed their intracellular localization, thus excluding the possibility that the more efficient delivery of encapsulated peptide or protein to the DCs was due to cell surface-adherent microparticles creating high local concentrations at the cell membrane. Our data support the view that these microparticles, having diameters of <10 μm, were taken up by phagocytosis (Tabata, Y., and Y. Ikada. 1990. Phagocytosis of polymer microspheres by macrophages. Adv. Polymer. Sci. 94:107; incorporated herein by reference). The exact molecular events surrounding microparticle phagocytosis, and whether they are identical for differing particle types, are not completely understood, although the identification and targeting or molecules involved in phagocytosis are an area of active research interest (Bonifaz, L. C., D. P. Bonnyay, A. Charalambous, D. I. Darguste, S. Fujii, H. Soares, M. K. Brimnes, B. Moltedo, T. M. Moran, and R. M. Steinman. 2004. In vivo targeting of antigens to maturing dendritic cells via the DEC-205 receptor improves T cell vaccination. J. Exp. Med. 199:815; incorporated herein by reference).

A concern in designing these pH-triggered particles was whether the polycationic polyamines would be cytotoxic at particle concentrations that were effective (Thomas, T., S. Balabhadrapathruni, M. A. Gallo, and T. J. Thomas. 2002. Development of polyamine analogs as cancer therapeutic agents. Oncol. Res. 13:123; incorporated herein by reference). In vitro, microparticles did not cause significant apoptosis in DCs after overnight incubation, even though microscopy showed DCs to have engulfed significant numbers of particles per cell. Toxicity was minimal at particle concentrations that effectively loaded peptide and protein into DCs. Moreover, the functional properties and phenotype of DCs loaded with microparticles were not altered.

The delivery of peptide antigen to human DCs by pH-triggered microparticles resulted in robust stimulation of antigen-specific T cells in vitro, which was significantly greater than that caused by nontriggering poly-HEME microparticles, or soluble peptide. DCs loaded with microparticles showed no increase in expression of costimulatory molecules. Thus, the increase T cell stimulation in vitro was not due simply to maturation of the DCs with global enhancement of its ability to activate T cells. Rather, the enhanced T cell stimulation may have been due to increased and possibly prolonged presentation of the antigen. Recent data have shown that the phagosome contains components of the endoplasmic reticulum that are essential for antigen presentation, such as TAP and MHC class I (Guermonprez, P., L. Saveanu, M. Kleijmeer, J. Davoust, P. Van Endert, and S. Amigorena. 2003. ER-phagosome fusion defines an MHC class I cross-presentation compartment in dendritic cells. Nature 425:397; Houde, M., S. Bertholet, E. Gagnon, S. Brunet, G. Goyette, A. Laplante, M. F. Princiotta, P. Thibault, D. Sacks, and M. Desjardins. 2003. Phagosomes are competent organelles for antigen cross-presentation. Nature 425:402; Ackerman, A. L., C. Kyritsis, R. Tampe, and P. Cresswell. 2003. Early phagosomes in dendritic cells form a cellular compartment sufficient for cross presentation of exogenous antigens. Proc. Natl. Acad. Sci. USA 100:12889; each of which is incorporated herein by reference). This suggests that the phagosome itself plays a direct role in the cross-presentation of exogenous antigen by MHC class I. Targeting the release of MHC class I antigens directly to the phagosome by pH-triggered microparticles may account, in part, for the increase antigen presentation seen with the microparticles compared with soluble peptide that enters the cell by pinocytosis. This might constitute an advantage over nanoparticulate formulations, such as liposomes, which are small enough to be taken up by pino- or endocytosis (Nair, S., A. M. Buiting, R. J. Rouse, N. Van Rooijen, L. Huang, and B. T. Rouse. 1995. Role of macrophages and dendritic cells in primary cytotoxic T lymphocyte responses. Int. Immunol. 7:679; Agrawal, A. K., and C. M. Gupta. 2000. Tuftsin-bearing liposomes in treatment of macrophage-based infections. Adv. Drug Delivery Rev. 41:135; Ignatius, R., K. Mahnke, M. Rivera, K. Hong, F. Isdell, R. N. Steinman, M. Pope, and L. Stamatatos. 2000. Presentation of proteins encapsulated in sterically stabilized liposomes by dendritic cells initiates CD8⁺ T-cell responses in vivo. Blood 96:3505; Oussoren, C., J. Zuidema, D. J. Crommelin, and G. Storm. 1997. Lymphatic uptake and biodistribution of liposomes after subcutaneous injection. II. Influence of liposomal size, lipid composition and lipid dose. Biochim. Biophys. Acta 1328:261; incorporated herein by reference). Although liposomes have been used previously to improve CTL, priming in vitro (Reddy, R., F. Zhou, L. Huang, F. Carbone, M. Bevan, and B. T. Rouse. 1991. pH sensitive liposomes provide an efficient means of sensitizing target cells to class I restricted CTL recognition of a soluble protein. J. Immunol. Methods 141:157; Reddy, R., F. Zhou, S. Nair, L. Huang, and B. T. Rouse. 1992. In vivo cytotoxic T lymphocyte induction with soluble proteins administered in liposomes. J. Immunol. 148:1585; each of which is incorporated herein by reference), microparticles described in this work are more likely to target phagocytic APCs as they did not enter nonphagocytic cells in detectable amounts.

Although the increased ability of E100 particles to stimulate T cells suggests that the pH-triggering capability is important for antigen presentation, we caution that pH sensitivity is not the only difference between E100 and poly-HEME. Both are polymethacrylates, but they are otherwise quite different molecules. The ideal control would have been a molecule very similar to E100, but not pH triggerable. However, E100 is a copolymer of three different methacrylate monomers, ˜50% of which are affected by pH. Because removing all pH triggerability would therefore involve altering a large fraction of the monomer units, there could not be a chemically identical (or very similar) molecule that did not pH trigger.

Because increasing the amount of Ag presented by DCs is thought to decrease the activation threshold for naïve T cells (Gett, A. V., F. Sallusto, A. Lanzavecchia, and J. Geginat. 2003. T cell fitness determined by signal strength. Nat. Immunol. 4:355; Wherry, E. J., K. A. Puorro, A. Porgador, and L. C. Eisenlohr. 1999. The induction of virus-specific CTL as a function of increasing epitope expression: responses rise steadily until excessively high levels of epitope are attained. J. Immunol. 163:3735; Kaech, S. M., and R. Ahmed. 2001. Memory CD8⁺ T cell differentiation: initial antigen encounter triggers a developmental program in naïve cells. Nat. Immunol. 2:415; Bullock, T. N., D. W. Mullins, and V. H. Engelhard. 2003. Antigen density presented by dendritic cells in vivo differentially affects the number and avidity of primary, memory, and recall CD8⁺ T cells. J. Immunol. 170:1822; Badovinac, V. P., B. B. Porter, and J. T. Harty. 2002. Programmed contraction of CD8⁺ T cells after infection. Nat. Immunol. 3:619; Langenkamp, A., G. Casorati, C. Garavaglia, P. Dellabona, A. Lanzavecchia, and F. Sallusto. 2002. T cell priming by dendritic cells: thresholds for proliferation, differentiation and death and intraclonal functional diversification. Eur. J. Immunol. 32:2046; each of which is incorporated herein by reference), we wished to determine whether the microparticles resulted in improved priming of naïve T cells. HHD mice are naïve to the M58 epitope, but have an immunodominant T cell response to M58 after immunization with whole influenza virus (Pascolo, S., N. Bervas, J. M. Ure, A. G. Smith, F. A. Lemonnier, and B. Perarnau. 1997. HLA-A2.1-restricted education and cytolytic activity of CD8⁺ T lymphocytes from β₂ microglobulin (β₂m) HLA-A2.1 monochain transgenic H-2Db β₂m double knockout mice. J. Exp. Med. 185:2043; incorporated herein by reference). HHD mice offered the opportunity to evaluate T cell priming in complex cellular environment that would be as close to the human setting as possible. In vivo, we found that vaccinating HHD mice with particles encapsulating a MHC I epitope resulted in CTL priming, and was much more effective than vaccination with soluble peptide. This finding might not have been predicted by our in vitro data, which showed that phagocytosis of particles by DCs was not associated with activation/maturation of DCs, and by the fact that the vaccine contained no helper epitopes that would have allowed antigen-specific CD4⁺ cells to activate/mature antigen-loaded DCs. However, like many microparticulate formulations, injection of the pH-triggered microparticles induces transient, mild inflammation at the vaccine site (Kohane, D. S., D. G. Anderson, C. Yu, and R. Langer. 2003. pH-triggered release of macromolecules from spray-dried polymethacrylate microparticles. Pharm. Res. 20:1533; each of which is incorporated herein by reference). It is possible that local release of inflammatory cytokines and chemokines may have induced activation of APCs. In this setting, the combination of local inflammation and increased antigen presentation on APCs may allow naïve T cells to be primed efficiently.

Although the present formulation of pH-triggered microparticles allowed T cell priming in vivo, the current formulation could be improved for vaccine use if it caused DC maturation. Coencapsulation of immunomodulatory reagents with the Ag of choice should be possible (Thomas, T. T., D. S. Kohane, A. Wang, and R. Langer. 2004. Microparticulate formulations for the controlled release of interleukin-2. J. Pharm. Sci. 93:1100; each of which is incorporated herein by reference). For instance, CpG oligonucleotides are potent activators of the innate immune system, and recent data suggest that their cognate receptor, TLR 9, interacts with CpG-bearing motifs in the endosomal compartment, presumably to permit DCs to scan for DNA from invading microorganisms that have been phagocytosed (Guermonprez, P., L. Saveanu, M. Kleijmeer, J. Davoust, P. Van Endert, and S. Amigorena. 2003. ER-phagosome fusion defines an MHC class I cross-presentation compartment in dendritic cells. Nature 425:397; Houde, M., S. Bertholet, E. Gagnon, S. Brunet, G. Goyette, A. Laplante, M. F. Princiotta, P. Thibault, D. Sacks, and M. Desjardins. 2003. Phagosomes are competent organelles for antigen cross-presentation. Nature 425:402; Ackerman, A. L., C. Kyritsis, R. Tampe, and P. Cresswell. 2003. Early phagosomes in dendritic cells form a cellular compartment sufficient for cross presentation of exogenous antigens. Proc. Natl. Acad. Sci. USA 100:12889; each of which is incorporated herein by reference). Coencapsulation of CpG oligonucleotides along with antigen in pH-triggered microparticles would therefore allow the efficient delivery of an activating ligand to a compartment rich in its receptors and may significantly improve the ability of the microparticles to prime a long-lasting T cell response in vivo.

Another interesting related application is made possible by the ease with which particle density can be modified (Kohane, D. S., M. Lipp, R. Kinney, D. Anthony, N. Lotan, and R. Langer. 2002. Biocompatibility of lipid-protein-sugar particles containing bupivacaine in the epineurium. J. Biomed. Mater. Res. 59:450; incorporated herein by reference), so that formulations could be used for inhalation delivery (Ben-Jebria, A., D. Chen, M. L. Eskew, R. Vanbever, R. Langer, and D. A. Edwards. 1999. Large porous particles for sustained protection from carbachol-induced bronchoconstriction in guinea pigs. Pharm. Res. 16:555; incorporated herein by reference), which might render them useful for induction of airway mucosal immunity. Microparticle production is relatively straightforward once an appropriate formulation has been developed, and is easily amenable to scale-up.

Improving the CD8⁺ T cell response to vaccine requires the optimization of several factors, including epitope choice, antigen delivery, and DC maturation pH-triggered microparticles capitalize on the physiology of exogenous antigen entry into the MHC I pathway and improve one critical component of the initiation of the T cell response: antigen presentation. The particles represent a flexible platform on which to base future vaccine designs to elicit CD8⁺ immunity to cancer and infectious diseases.

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A microparticle comprising at least one agent to be delivered, a pH triggering agent, and a polymer, wherein the polymer is selected from the group of polymethacrylates and polyacrylates.

2. A microparticle comprising at least one agent to be delivered, a pH triggering agent, and at least two components selected from the group consisting of lipids, proteins, sugars, and polymers.

3. The microparticle of claim 2 comprising at least one agent to be delivered, a pH triggering agent, and at least three components selected from the group consisting of lipids, proteins, sugars, and polymers.

4. The microparticle of claim 2 comprising at least one agent to be delivered, a pH triggering agent, a lipid, a protein, a sugar, and a polymer.

5. The microparticle of claim 2 comprising at least one agent to be delivered, a pH triggering agent, a polymer, and at least one component selected from the group consisting of lipids, proteins, and sugars.

6. The microparticle of claim 2 comprising at least one agent to be delivered and a pH triggering agent, wherein the agent is encapsulated in a lipid-protein-sugar matrix.

7. The microparticle of claim 2 comprising at least one agent to be delivered and a pH triggering agent, wherein the agent is encapsulated in a lipid-protein matrix.

8. The microparticle of claim 1, wherein the pH triggering agent is an acid soluble polymer.

9. The microparticle of claim 1, wherein the pH triggering agent is selected from the group consisting of small molecules, ortho-esters, polymers, proteins, peptides, lipids, synthetic polymers, phospholipids, cationic proteins, polyacrylates, polymethacrylates, poly(beta-amino esters), and acid soluble polymers.

10. The microparticle of claim 1, wherein the pH triggering agent is a lipid or phospholipid.

11. The microparticle of claim 1, wherein the agent is selected from the group consisting of protein, peptide, polynucleotide, organic molecule, drug, and small molecule.

12. The microparticle of claim 1, wherein the agent is an antigen.

13. The microparticle of claim 1, wherein the agent is an antigenic protein.

14. The microparticle of claim 1, wherein the agent is a polynucleotide encoding an antigenic protein.

15. The microparticle of claim 1, wherein the pH triggering agent is a polymethacrylate.

16. The microparticle of claim 1, wherein the pH triggering agent is soluble in an aqueous solution of pH less than 7.

17. The microparticle of claim 1, wherein the pH triggering agent is soluble in an aqueous solution of pH less than 6.

18. The microparticle of claim 1, wherein the pH triggering agent is soluble in an aqueous solution of pH less than 5.

19. The microparticle of claim 1, wherein the pH triggering agent is a cationic protein at pH 7.4.

20. The microparticle of claim 1, wherein the pH triggering agent is poly(butyl methacrylate-co-(2-dimethylaminoethyl) methacrylate-co-methyl methacrylate (1:2:1) (Eudragit E100).

21. The microparticle of claim 1, wherein the percentage of pH triggering agent in the microparticle ranges from 1% to 80%.

22. The microparticle of claim 1, wherein the percentage of pH triggering agent in the microparticle ranges from 5% to 50%.

23. The microparticle of claim 1, wherein the percentage of pH triggering agent in the microparticle ranges from 10% to 40%.

24. The microparticle of claim 1, wherein the percentage of pH triggering agent in the microparticle is approximately 20%.

25. The microparticle of claim 1, wherein the percentage of pH triggering agent in the microparticle is at least 20%.

26. The microparticle of claim 1, wherein the density of the microparticle is between 0.3 g/ml and 0.1 g/ml.

27. The microparticle of claim 1, wherein the microparticle is approximately 1 to 10 microns in diameter.

28. The microparticle of claim 1, wherein the microparticle is approximately 2 to 4 microns in diameter.

29. The microparticle of claim 2, wherein the polymer is selected from the group consisting of polyesters, polyamides, polycarbonates, polycarbamates, polyacrylates, polymethacrylates, polystyrenes, polyureas, polyether, polythioethers, glycols, and polyamines.

30. The microparticle of claim 1, wherein the polymer is biocompatible and biodegradable.

31. A pharmaceutical composition comprising pH triggered microparticles, wherein the microparticles comprise at least one agent to be delivered, a polymer, and a pH triggering agent.

32. A pharmaceutical composition comprising pH-triggered microparticles of at least one agent encapsulated in a matrix comprising a lipid, a protein, and a pH triggering agent.

33. The pharmaceutical composition of claim 31, wherein the pH triggering agent is an acid soluble polymer.

34. The pharmaceutical composition of claim 32, wherein the pH-triggered microparticles further comprise a sugar.

35. The pharmaceutical composition of claim 31, wherein the agent is selected from the group consisting of protein, peptide, polynucleotide, organic molecule, drug, and small molecule.

36. The pharmaceutical composition of claim 31, wherein the agent is an antigen.

37. The pharmaceutical composition of claim 32, wherein the lipid is dipalmitoylphosphatidylcholine (DPPC).

38. The pharmaceutical composition of claim 31, wherein the pH triggering agent is a polymethacrylate.

39. The pharmaceutical composition of claim 31, wherein the pH triggering agent is soluble in an aqueous solution of pH less than 7.

40. The pharmaceutical composition of claim 31, wherein the pH triggering agent is soluble in an aqueous solution of pH less than 6.

41. The pharmaceutical composition of claim 31, wherein the pH triggering agent is soluble in an aqueous solution of pH less than 5.

42. The pharmaceutical composition of claim 31, wherein the pH triggering agent is a cationic protein at pH 7.4.

43. The pharmaceutical composition of claim 31, wherein the pH triggering agent is poly(butyl methacrylate-co-(2-dimethylaminoethyl) methacrylate-co-methyl methacrylate (1:2:1) (Eudragit E110).

44. The pharmaceutical composition of claim 32, wherein the protein is albumin.

45. The pharmaceutical composition of claim 34, wherein the sugar is lactose.

46. The pharmaceutical composition of claim 31, wherein the percentage of pH triggering agent in the microparticles ranges from 1% to 80%.

47. The pharmaceutical composition of claim 31, wherein the percentage of pH triggering agent in the microparticles ranges from 5% to 50%.

48. The pharmaceutical composition of claim 31, wherein the percentage of pH triggering agent in the microparticles ranges from 10% to 40%.

49. The pharmaceutical composition of claim 31, wherein the percentage of pH triggering agent in the microparticles is approximately 20%.

50. The pharmaceutical composition of claim 31, wherein the percentage of pH triggering agent in the microparticles is at least 20%.

51. The pharmaceutical composition of claim 31, wherein the density of the microparticles ranges from 0.3 g/ml to 0.1 g/ml.

52. The pharmaceutical composition of claim 31, wherein the microparticles are approximately 1 to 10 microns in diameter.

53. The pharmaceutical composition of claim 31, wherein the microparticles are approximately 2 to 4 microns in diameter.

54. The pharmaceutical composition of claim 31 further comprising an adjuvant.

55. The pharmaceutical composition of claim 54, wherein the adjuvant is selected from the group consisting of lipids, proteins, DNA, DNA-protein, DNA-RNA hybrids, lipoproteins, aptamers, and antibodies.

56. A method of administering a pH-triggered microparticles, the method comprising steps of:
providing a patient;
providing a pharmaceutical composition comprising pH-triggered microparticles comprising at least one agent, a protein, a lipid, and a pH triggering agent;
administering the pharmaceutical composition to the patient.

57. The method of claim 56, wherein the step of administering comprises administering the composition parenterally.

58. The method of claim 56, wherein the step of administering comprises administering the composition inhalationally.

59. The method of claim 56, wherein the step of administering comprises administering the composition orally.

60. The method of claim 56, wherein the step of administering comprises administering the composition to a mucosal surface of the patient.

61. The method of claim 56, wherein the step of administering comprises administering the composition to the skin of the patient.

62. The method of claim 56, wherein the step of administering results in intracellular delivery of the agent to be delivered.

63. The method of claim 56, wherein the agent is an antigen.

64. A method of transfection, the method comprising steps of:
providing at least one cell;
providing a composition comprising pH-triggered microparticles comprising a polynucleotide, a protein, a lipid, and a pH triggering agent;
contacting the cell with the composition to achieve transfection of the polynucleotide into the cell.

65. A method of immunizing a patient, the method comprising steps of:
providing a patient to be immunized;
providing a pharmaceutical composition comprising an antigen encapsulated in a matrix of lipid, protein, and pH triggering agent;
administering an effective amount of the pharmaceutical composition to the patient to stimulate an immune response.

66. The method of claim 65, wherein the antigen is a protein.

67. The method of claim 65, wherein the step of administering comprises administering the composition to a mucosal surface of the patient.

68. A method of treating a patient in need of gene therapy, the method comprising steps of:
providing a patient to be treated;
providing a pharmaceutical composition comprising a polynucleotide encapsulated in a matrix of lipid, protein, and pH triggering agent;
administering an effective amount of the pharmaceutical composition to the patient to result in transfection of at least one cell of the patient.

69. A method of preparing pH-triggered microparticles, the method comprising steps of:
providing an agent;
contacting the agent with a pH triggering agent and at least one component selected from the group consisting of lipids, proteins, sugars, and polymers; and
spray-drying resulting mixture to create microparticles.

70. The method of claim 69, wherein the step of contacting comprises contacting the agent with a pH triggering agent and at least two components selected from the group consisting of lipids, proteins, sugars, and polymers.

71. The method of claim 69, wherein the step of contacting comprises contacting the agent with a pH triggering agent and at least three components selected from the group consisting of lipids, proteins, sugars, and polymers.

72. The method of claim 69, the method comprising steps of:
providing an agent;
contacting the agent with a mixture of a lipid, a protein, and pH triggering agent; and
spray drying resulting mixture to create microparticles.
